(19) United States
(12) Patent Application Publication
(10) Pub. No.: US 2002/0155473 A1
(43) Pub. Date: Oct. 24, 2002

(54) Methods for identifying G-protein coupled receptors associated with diseases

(76) Inventors: Zaoyuan Peng, Palo Alto, CA (US); Zailin Yu, San Leandro, CA (US); Andrew Wiley, Napa, CA (US); Qianjin Hu, Castro Valley, CA (US)

Correspondence Address: MORRISON & FOERSTER LLP, 755 PAGE MILL RD, PALO ALTO, CA, 94304-1018, US

(21) Appl. No.: 10/032,106

(22) Filed: Dec. 21, 2001

Related U.S. Application Data
(60) Provisional application No. 60/258,070, filed on Dec. 22, 2000.

(51) Int. Cl. C12Q 1/68

(52) U.S. Cl. 435/6

(57) ABSTRACT

The present invention describes a method for isolating novel G-protein coupled receptors (GPCRs) whose expression is associated with disease states. The invention provides polynucleotide and polypeptide sequences whose expression is associated with neurodegenerative diseases and cancer. The invention also provides evidence of novel G-protein coupled receptors (GPCRs) related polynucleotide sequences whose expression level is associated with Parkinson's disease, Alzheimer's disease and leukemia. The present invention provides methods for identifying GPCR polypeptides associated with diseases and provides diagnostic compositions and methods for the detection of diseases.

### CROSS-REFERENCE TO RELATED APPLICATION

This application claims the benefit of U.S. patent application Serial No. 60/258,070, filed on Dec. 22, 2000, which is hereby incorporated by reference in its entirety.

### TECHNICAL FIELD OF THE INVENTION

The present invention relates to the field of detection of gene expression. In particular, the present invention relates to methods for identifying novel genes whose expression is associated with disease states. More particularly, the present invention relates to novel GPCR polynucleotides and polypeptides associated with CNS diseases and cancer.

### BACKGROUND

G-protein linked cell receptors comprise the largest superfamily of mammalian proteins with more than 1,000 different members identified to date. (Gether, U. Endocr. Rev. 21(1):90-113 (Feb 2000)). GPCRs are found in a very wide range of species, and are often involved in signal transduction across cell membranes. GPCR sequences include a characteristic seven hydrophobic transmembrane domains which span the plasma membrane and form a bundle of antiparallel alpha helices, commonly identified as seven transmembrane (7-TM) helices. The transmembrane domains account for structural and functional features of the receptor. Ligands which activate the various members of the GPCR family include an enormous variety of molecules such as amines, amino acids and peptides as well as several small hydrophobic molecules. Usually the 7-TM bundle of helices forms a binding pocket. When the binding site needs to accommodate bulky molecules, the extracellular N-terminal segment or one or more of the three extracellular loops also participate in binding and in subsequent induction of conformational change in intracellular portions of the receptor. The activated receptor, in turn, interacts with an intracellular heterotrimeric G-protein complex which mediates further intracellular signaling activities, such as interactions with guanine nucleotide binding (G) proteins and the production of second messengers such as cyclic AMP (cAMP), phospholipase C, inositol triphosphate or ion channel proteins (Baldwin, J. M. (1994) Curr. Opin. Cell Biol. 6:180-190). GPCRs may also function via other activation mechanisms such as, photon-activation of opsins and self-activation of thrombin receptor after cleavage of its N-terminus by thrombin. (see, Watson, S. and S. Arkinstall (1994) The G-Protein Linked Receptor Facts Book, Academic Press, San Diego, Calif.).

Among membrane-bound receptors, the 7-TM receptors are the most abundant, comprising up to 1% of the genome. They transduce signals in response to a large number of physiologically important molecules, including photons, organic odorants, nucleotides, nucleosides, biogenic amines, sugars, peptides, lipids, proteins and viruses. They also catalyze GDP/GTP nucleotide exchange on heterotrimeric G-proteins and therefore are also referred to as G-protein-coupled receptors (GPCRs). To date, greater than 60% of the 400 or so drug targets are GPCRs and these receptors play a key role in homeostatic regulation in many tissues.

The amino-terminus of a GPCR is extracellular, of variable length and often glycosylated, while the carboxy-terminus is cytoplasmic and generally phosphorylated. Extracellular loops of GPCRs alternate with intracellular loops and link the transmembrane domains. The most conserved domains of GPCRs are the transmembrane domains and the first two cytoplasmic loops. GPCRs range in size from under 400 to over 1000 amino acids (Coughlin, S. R. (1994) Curr. Opin. Cell Biol. 6:191-197).

It is generally believed that members of the 7-TM helix G protein-coupled receptor family pair up with their own kind but do not bind to other family members (Hebert, T. E. and Bouvier, M., Biochem. Cell Biol. 76, 1 (1998)). However, recent studies provide evidence that GPCRs can pair up with even rather distantly related relatives to form heterodimeric receptors with distinct properties (Rocheville, M. et al., Science 288, 154-157 (2000)).

A highly conserved D-R-Y motif comprising the amino acids Asp-Arg-Tyr is also characteristic of GPCRs (Acharya, S., and Karnik, S. S. J. Biol. Chem. 271:25406-25411 (1996)) and is involved in constitutive activity and structural stability of GPCRs. (see, Alewijnse, A. E. et al., Mol Pharmacol 57(5):890-898 (2000)).

GPCRs respond to a diverse array of ligands including lipid analogs, amino acids and their derivatives, peptides, cytokines, and specialized stimuli such as light, taste, and odor. GPCRs function in physiological processes including vision (the rhodopsins), smell (the olfactory receptors), neurotransmission (muscarinic acetylcholine, dopamine, and adrenergic receptors), and hormonal response (luteinizing hormone and thyroid-stimulating hormone receptors).

GPCR mutations, affecting both loss-of-function and activation, have been associated with numerous human diseases (Coughlin, supra). For instance, retinitis pigmentosa may arise from either loss-of-function or activating mutations in the rhodopsin gene. Somatic activating mutations in the thyrotropin receptor cause hyperfunctioning thyroid adenomas (Parma, J. et al. (1993) Nature 365:649-651). Parma et al. also suggest that certain GPCRs susceptible to constitutive activation may be proto-oncogenes.

Neuropeptide Y (NPY), pancreatic polypeptide (PP), and peptide YY (PYY) are structurally related peptides found in higher vertebrates. NPY is produced in the central and peripheral nervous systems. NPY plays a role in the stimulation of food intake, anxiety, facilitation of learning and memory, and regulation of the neuroendocrine and cardiovascular systems. NPY also stimulates vascular smooth muscle contraction, modulates hormone secretion, and has been implicated in the pathophysiology of hypertension, congestive heart failure, affective disorders and appetite regulation (Watson, S. and S. Arkinstall (1994) The G-Protein Linked Receptor Facts Book, Academic Press, San Diego Calif., pp. 194-198). PP is produced in endocrine cells in the pancreas and inhibits pancreatic secretion, gall bladder contraction, and gut motility. PYY is produced in endocrine cells of the pancreas and large intestine. PYY has actions similar to those of PP, and in addition redistributes blood flow in gut vessels. Both PP and PYY are released into the circulation in response to food intake. These structurally related peptides accomplish their varied biological functions through interaction with distinct GPCR subtypes. Several receptor subtypes have been defined by their ability to bind NPY, PYY, PP, and derivatives of these peptides. At least five distinct receptor subtypes have been characterized to date (Weinberg, D. H. et al. (1996) J. Biol. Chem. 271:16435-16438).

Cholecystokinin (CCK) is a bioactive peptide which is present throughout the digestive tract, and is also found in smooth muscle tissues such as bladder and uterus, in secretory glands such as exocrine pancreas, and in the brain. The major physiological actions of CCK are gall bladder contraction, pancreatic enzyme secretion, and regulation of secretion/absorption in the gastrointestinal tract. CCK receptors are GPCRs found in peripheral tissues including pancreas, stomach, intestine and gall bladder, and in limited amounts in the brain. CCK receptors mediate pancreatic acinar secretion and gallbladder contraction (De Weerth, A. et al. (1993) Am. J. Physiol. 265:G1116-G1121). The CCK-A receptor has been implicated in the pathogenesis of schizophrenia, Parkinson's disease, drug addiction and feeding disorders (Watson and Arkinstall, supra, pp. 89-95).

### SUMMARY OF THE INVENTION

The present invention describes a method for isolating novel G-protein coupled receptors (GPCRs) whose expression is associated with diseases. The present invention relates to novel GPCR polypeptides associated with CNS diseases and cancer and antibodies specific for the GPCR polypeptides in the detection, diagnosis, prevention and/or treatment of the GPCR expression-related diseases.

The present invention relates to materials and methods for diagnosing, preventing and/or treating GPCR expression-related diseases in subjects. The present invention is based, in part, on the discovery of differential levels of expression of GPCR-related polynucleotides in tissue samples of Parkinson's disease, Alzheimer's disease and leukemia patients, as compared to normal tissue samples. The present invention provides polynucleotides, as well as their corresponding GPCR gene products, that are present at elevated levels in these disease states. These polynucleotides (or fragments thereof) and polypeptides (or antigenic fragments thereof), and antibodies that bind such polypeptides, are useful in a variety of diagnostic, prophylactic and therapeutic methods.

The present invention provides a novel GPCR polynucleotide having the sequence illustrated in FIG. 1B (SEQ ID NO 1) for use in the detection, diagnosis, prevention and treatment of Alzheimer's diseases and leukemia.

The present invention provides a novel GPCR polynucleotide having the sequence illustrated in FIG. 2B (SEQ ID NO 2) for use in the detection, diagnosis, prevention and treatment of Parkinson's and Alzheimer's diseases and leukemia.

The present invention provides a novel GPCR polynucleotide having the sequence illustrated in FIG. 3B (SEQ ID NO 3) for use in the detection, diagnosis, prevention and treatment of Parkinson's and Alzheimer's diseases and leukemia.

The present invention provides a novel GPCR polynucleotide having the sequence illustrated in FIG. 4B (SEQ ID NO 4) for use in the detection, diagnosis, prevention and treatment of Parkinson's and Alzheimer's diseases and leukemia.

The present invention provides a novel GPCR polynucleotide having the sequence illustrated in FIG. 5B (SEQ ID NO 5) for use in the detection, diagnosis, prevention and treatment of leukemia.

A method is provided for identifying a gene whose expression level is associated with a disease state, the method comprising: identifying at least one gene having a nucleic acid sequence encoding a protein comprising a physical characteristic; selecting a polynucleotide sequence from the nucleic acid sequence, wherein the polynucleotide sequence is specific for a protein comprising the physical characteristic; detecting a level of expression of the polynucleotide sequence or a complement thereof in a diseased tissue sample; detecting a level of expression of the polynucleotide sequence or a complement thereof in a normal tissue sample; and comparing the level of expression of the polynucleotide sequence or a complement in the diseased tissue sample to a level of expression of the gene in the control tissue sample, wherein an altered level of expression of the polynucleotide sequence or a complement in the diseased tissue sample correlates with the disease state.

The physical characteristic may comprises seven transmembrane (7-TM) domains, or an Asp-Arg-Tyr (DRY) motif, or a signal peptide sequence characteristic of a secreted protein, or a signal peptide sequence characteristic of a mitochondrial protein, or an amino acid sequence characteristic of a structural feature of the protein, or an amino acid sequence characteristic of a function of the protein.

The identification of the gene may comprise searching a nucleic acid sequence database for nucleic acid sequences which encode a protein comprising the physical characteristic. The nucleic acid sequence database is optionally an electronic library. The gene is optionally identified using a search algorithm. The identification of the gene may further comprise selecting at least one gene whose expression is known to correlate with a disease state. In one embodiment, the gene is a novel G-protein linked cell receptor type gene

In one embodiment of the method, the detection of the level of expression of the polynucleotide sequence comprises: selecting at least one isolated oligonucleotide comprising the polynucleotide sequence or a fragment thereof, contacting the oligonucleotide with a nucleic acid preparation from the tissue sample; and detecting a level of expression of the polynucleotide sequence by detecting an amount of hybridization of the nucleic acid preparation to the oligonucleotide under stringent conditions.

In one embodiment, the oligonucleotide is attached to a solid support. In another embodiment, the solid support is a microarray.

In one embodiment of the method, the selection of the polynucleotide sequence comprises determining at least one of a set of factors comprising (i) a redundancy of the sequence, (ii) an efficiency of hybridization to a complementary sequence, and (iii) a likelihood of the polynucleotide sequence comprising an intron.

The nucleic acid preparation from the tissue sample may comprise a detectable label. The detectable label is optionally selected from the group consisting of a fluorescent label, an enzymatic label, a chemiluminescent label, a colorimetric label, and a radioactive label. The nucleic acid preparation from the tissue sample is optionally amplified before detection. In one embodiment, the amplification is a conducted by a polymerase chain reaction (PCR). In one embodiment, the amplification is a conducted by a quantitative polymerase chain reaction (QPCR).

In one embodiment of a method according to the invention, comparing the level of expression of the gene comprises: providing at least one isolated oligonucleotide comprising the polynucleotide sequence or a fragment thereof; contacting the oligonucleotide with an amount of nucleic acid preparation from a disease tissue sample; contacting the oligonucleotide with an equal amount of nucleic acid preparation from a normal tissue sample; and comparing the level of expression of the polynucleotide sequence in the tissue samples by detecting an amount of hybridization of each nucleic acid preparation to the oligonucleotide under stringent conditions.

In some embodiments, the polynucleotide is attached to a solid support, wherein the solid support is optionally microarray. The nucleic acid preparation is optionally an RNA preparation, wherein the RNA preparation may be further processed to generate a labeled nucleic acid probe. The labeled nucleic acid probe may comprise a label coupled to the probe, wherein the label is selected from the group consisting of a biotin, an avidin, a streptavidin, an antibody, an antigen, a peptide, a fluorescent label, an enzymatic label, a chemiluminescent label, a colorimetric label, and a radioactive label.

The invention also provides a method for detecting in a cell an expression of a gene whose expression level is associated with a disease state, the method comprising: cloning a polynucleotide fragment comprising a sequence of the cloned gene in an expression vector; and detecting a corresponding protein in a cell transformed with the vector comprising the cloned fragment. In some embodiments the protein is detected by an antibody, or a monoclonal antibody.

The invention further provides a method for preparing an antibody specific for a polypeptide product of a gene whose expression level is associated with a disease state, the method comprising: cloning a polynucleotide fragment comprising a sequence of the cloned gene in an expression vector; isolating a polypeptide expressed by the vector, wherein the polypeptide comprises an amino acid sequence corresponding to the cloned polynucleotide; immunizing an animal with the isolated polypeptide; and isolating anti-peptide antibodies specific for the isolated polypeptide from the immunized animal.

The invention provides an isolated novel polynucleotide comprising a gene whose expression level is associated with a disease state, the polynucleotide comprising a nucleic acid sequence encoding a protein comprising a physical characteristic, wherein the polynucleotide or a fragment thereof is differentially expressed in a diseased tissue sample as compared to a normal tissue sample.

The isolated polynucleotide may comprise a nucleic acid sequence which encodes a protein comprising at least one of the characteristics of: (a) seven transmembrane (7-TM) domains, (b) an amino acid sequence comprising an Asp-Arg-Tyr (DRY) motif and (c) a signal peptide. The isolated polynucleotide may comprise a detectable label. The isolated polynucleotide, or fragment thereof, is optionally attached to a solid support. The isolated polynucleotide may be single or double stranded.

The invention further provides a host cell, an array, a composition comprising a test cell sample and an electronic library, each comprising at least one isolated polynucleotide of the invention.

The present invention provides a method for the detection and diagnosis of diseases, the method comprising detecting an altered level of expression of the GPCR polynucleotides comprising sequences shown in FIGS. 1B, 2B, 3B, 4B and 5B (SEQ ID NOS 1-5). In one aspect of the invention, a diagnosis of Parkinson's disease, Alzheimer's disease and/or leukemia correlates to an altered level of expression of the polynucleotides comprising sequences shown in FIGS. 1B, 2B, 3B, 4B and 5B (SEQ ID NOS 1-5) in disease tissue samples. In one aspect of the invention, a diagnosis of Parkinson's and/or Alzheimer's disease correlates to a lower level of expression of the polynucleotides comprising sequences shown in FIGS. 1B, 2B, 3B, 4B and 5B (SEQ ID NOS 1-5) in patient tissue samples. In another aspect of the invention, a diagnosis of leukemia disease correlates to an elevated level of expression of the polynucleotides comprising sequences shown in FIGS. 1B, 2B, 3B, 4B and 5B in patient tissue samples.

The present invention also provides polynucleotides comprising a segment of at least 10 nucleotides in length which corresponds identically to a portion of the sequences shown in FIGS. 1B, 2B, 3B, 4B and 5B and whose expression correlates to a state of disease. In further embodiments, the segment of the polynucleotide sequence is up to at least 30, 40, 50, 60, 70, 80, 90, 100, 200, 300, 400, 500, 1000, or 1500 nucleotides in length.

In other embodiments, a polynucleotide of the present invention comprises a detectable label, and/or is attached to a solid support. In other embodiments, a polynucleotide of the present invention is single stranded and in yet other embodiments, is double stranded. The present invention also encompasses host cells comprising an isolated polynucleotide of the present invention.

In a further aspect, the present invention provides GPCR polypeptides comprising polypeptides having sequences shown in FIGS. 1C, 2C, 3C, 4C and 5C, or fragment thereof, whose presence and/or expression levels correlate to a state of central nervous system (CNS) disease, Parkinson's disease, Alzheimer's disease and leukemia. In one embodiment, the invention provides GPCR polypeptides having sequences shown in FIGS. 1C, 2C, 3C, 4C and 5C, or fragment thereof, whose presence and/or expression levels correlate to a state of Parkinson's and/or Alzheimer's disease and/or leukemia. In an additional embodiment, the polypeptide comprises a fragment that includes an epitope of the amino acid sequence shown in FIGS. 1C, 2C, 3C, 4C and 5C. In additional embodiments, the polypeptide or fragment thereof, is attached to a solid support.

The present invention also provides isolated antibodies or antigen binding fragments thereof, that bind to a polypeptide of the present invention. The present invention also provides isolated antibodies or antigen binding fragments thereof, that bind to a polypeptide encoded by a polynucleotide of the present invention. The present invention also provides isolated antibodies that bind to a polypeptide of the invention, or antigen binding fragment thereof, encoded by a polynucleotide made by the method comprising the following steps i)immunizing a host animal with a composition comprising said polypeptide of the present invention, or antigen binding fragment thereof, and ii) collecting cells from said host expressing antibodies against the antigen or antigen binding fragment thereof.

The present invention also provides isolated antibodies that bind to a polypeptide, or antigen binding fragment thereof, encoded by a polynucleotide of the present invention made by the method comprising the following steps: providing a cell line producing an antibody, wherein said antibody binds to a polypeptide of the present invention, or antigen binding fragment thereof, encoded by a polynucleotide of the present invention and culturing said cell line under conditions wherein said antibodies are produced. In additional embodiments, the antibodies are collected and monoclonal antibodies are produced using the collected host cells or genetic material derived from the collected host cells. In additional embodiments, the antibody is a polyclonal antibody. In a further embodiment, the antibody is attached to a solid surface or further comprises a detectable label.

The present invention also discloses a method for diagnosing a disease associated with a G-protein coupled receptor (GPCR) expression in a test tissue sample, the method comprising: detecting a level of expression of a polynucleotide comprising a GPCR DNA sequence or a fragment thereof; and comparing the level of expression of the polynucleotide in the test tissue sample to a level of expression in a control tissue sample, wherein an altered level of expression of the polynucleotide in the test tissue sample is indicative of a disease state.

The disease state may be selected from the group consisting of a central nervous system (CNS) disease, Parkinson's disease, Alzheimer's disease and leukemia.

The polynucleotide may comprise a GPCR DNA fragment at least 10 contiguous nucleotides in length. In some embodiments, the polynucleotide comprises: (a) a polynucleotide having sequences shown in FIGS. 1B, 2B, 3B, 4B and 5B (SEQ ID NOS 1-5), or its complement; (b) a fragment of the polynucleotide comprising the sequence shown in FIGS. 1B, 2B, 3B, 4B and SB (SEQ ID NOS 1-5), or its complement, wherein the fragment is at least 10 nucleotides in length; or (c) a polynucleotide that selectively hybridizes to the sequences shown in FIGS. 1B, 2B, 3B, 4B and 5B (SEQ ID NOS 1-5) or the fragment in (b).

The altered level of expression may be an elevated or a lowered level of expression.

The detecting optionally comprises measuring the level of an RNA comprising a sequence complementary to the polynucleotide, or its complement. The polynucleotide may comprise a detectable label. The detectable label is selected from the group consisting of a fluorescent label, an enzymatic label, a chemiluminescent label, a colorimetric label, and a radioactive label. The polynucleotide is optionally attached to a solid support, or a microarray. The polynucleotide may be single or double stranded. The polynucleotide may be amplified before detection. The amplification is optionally conducted by a conducted by polymerase chain reaction (PCR).

A lowered level of expression of the polynucleotide in the test tissue sample may be indicative of a CNS disease, a Parkinson's disease or a Alzheimer's disease. An elevated level of expression of the polynucleotide in the test tissue sample may be indicative of a leukemia disease.

The invention also provides methods for diagnosing a disease associated with a G-protein coupled receptor (GPCR) expression in a test tissue sample, the method comprising: detecting a level of expression of a polypeptide comprising sequences shown in FIGS. 1C, 2C, 3C, 4C and 5C (SEQ ID NOS 6-10), or a fragment thereof; and comparing the level of expression of the polypeptide in the test tissue sample to a level of expression in a control tissue sample, wherein an altered level of expression of the polypeptide in the test tissue sample is indicative of a disease state.

The disease state may be selected from the group consisting of a CNS disease, Parkinson's disease, Alzheimer's disease and leukemia. The polypeptide may comprise a fragment comprising at least one epitope of the amino acid sequences shown in FIGS. 1C, 2C, 3C, 4C and 5C (SEQ ID NOS 6-10). The polypeptide or fragment thereof, may be attached to a solid support or an array.

The invention further includes an isolated polynucleotide comprising: (a) a polynucleotide having sequences shown in FIGS. 1B, 2B, 3B, 4B and SB (SEQ ID NOS 1-5), or its complement; (b) a fragment of the polynucleotide having the sequence shown in FIGS. 1B, 2B, 3B, 4B and 5B (SEQ ID NOS 1-5), or its complement, wherein the fragment is at least 10 nucleotides in length; or (c) a polynucleotide that selectively hybridizes to the sequences shown in FIGS. 1B, 2B, 3B, 4B and 5B (SEQ ID NOS 1-5) or the fragment in (b), wherein expression of the isolated polynucleotide correlates to a state of disease.

The isolated polynucleotide may comprise a segment of up to at least 20, 30, 40, 50, 60, 70, 80, 90, 100, 200, 300, 400, 500, 1000 or 1500 nucleotides in length which corresponds identically to a portion of the sequences shown in FIGS. 1B, 2B, 3B, 4B and 5B (SEQ ID NOS 1-5).The isolated polynucleotide may further comprise a detectable label. The isolated polynucleotide may be attached to a solid support, be single or double stranded.

The invention further provides a host cell, an array comprising at least two polynucleotides according, a composition, comprising a test cell sample and an isolated polynucleotide, a kit for diagnosing a GPCR-related disease in a test sample, and an electronic library comprising at least one isolated polynucleotide comprising the sequences shown in FIGS. 1B, 2B, 3B, 4B and 5B (SEQ ID NOS 1-5).

The invention provides an isolated polypeptide encoded within a GPCR open reading frame, or a fragment thereof, whose expression levels in a tissue correlates to a disease state of the tissue. In some embodiments, the isolated polypeptide comprises the amino acid sequence encoded by the polypeptides shown in FIGS. 1C, 2C, 3C, 4C and 5C (SEQ ID NOS 6-10) or a fragment thereof. The disease state may be selected from the group consisting of central nervous system (CNS) disease, Parkinson's disease, Alzheimer's disease and leukemia. The polypeptide optionally comprises a fragment that includes an antigenic epitope comprising the amino acid sequence shown in FIGS. 1C, 2C, 3C, 4C and 5C (SEQ ID NOS 6-10).

The invention also provides an isolated antibody, or antigen binding fragments thereof, that bind to the polypeptide according to the invention. The antibody may be a polyclonal or monoclonal antibody, and may be attached to a solid surface and may comprise a detectable label.

The invention provides a method for producing an antibody, or antigen binding fragments thereof, that bind to a polypeptide according to the invention, the method comprising: (a) immunizing a host animal with a composition comprising the polypeptide, or fragment thereof; and (b) collecting one or more cells from said host, wherein said cells produce antibodies against the polypeptide or fragment thereof. In one embodiment the method further comprises: (c) producing monoclonal antibodies using the collected host cells or genetic material derived from the collected host cells.

The invention provides a method for screening a drug candidate for activity against a GPCR-related disease, the method comprising: (a) contacting a tissue sample derived from a cell associated with an altered GPCR-related disease with a drug candidate; (b) monitoring the expression in the tissue sample of a polynucleotide comprising the sequence shown in FIGS. 1B, 2B, 3B, 4B or 5B (SEQ ID NOS 1-5), or a complement thereof; and (c) determining the efficacy of the drug candidate.

The invention provides a method for detecting a disease associated with expression of a GPCR polypeptide in a test cell sample, the method comprising: (a) detecting a level of expression of at least one polypeptide, or a fragment thereof, according to the invention; and (b) comparing said level of expression of the polypeptide in the test sample with a level of expression of polypeptide in the control cell sample, wherein an altered level of expression of the polypeptide in the test cell sample relative to the level of expression of the polypeptide in the control cell sample is indicative of the presence of the disease in the test cell sample.

The invention provides a method for detecting a disease associated with the presence of an anti-GPCR antibody in a test cell sample, the method comprising: (a) detecting a level of an antibody against a polypeptide, or fragment thereof, according to the invention; and (b) comparing said level of said antibody in the test sample with a level of said antibody in the control cell sample, wherein an altered level of antibody in said test cell sample relative to the level of antibody in the control cell sample is indicative of the presence of the disease in the test cell sample.

The invention provides a method for stimulating an immune response in a human against cells that express GPCR polypeptides, the method comprising administering to a human an immunogenic amount of: (a) a polypeptide, comprising an amino acid sequence of a GPCR polypeptide according to the invention; or (b) a mutein or variant of a polypeptide comprising the amino acid sequence of a GPCR polypeptide according to the invention.

The present invention also provides compositions comprising a test cell sample and an isolated polynucleotide of the present invention. The present invention further provides methods for detecting disease associated with expression of a polypeptide in a test cell sample, comprising the steps of: i) detecting a level of expression of at least one polypeptide of the present invention, or a fragment thereof and ii) comparing said level of expression of the polypeptide in the test sample with a level of expression of polypeptide in the control cell sample, wherein an altered level of expression of the polypeptide in the test cell sample relative to the level of expression of the polypeptide in the control cell sample is indicative of the presence of the disease in the test cell sample. The present invention also provides methods for detecting disease associated with the presence of an antibody in a test cell sample, comprising the steps of: i) detecting a level of an antibody of the present invention, and ii) comparing said level of said antibody in the test sample with a level of said antibody in the control cell sample, wherein an altered level of antibody in said test cell sample relative to the level of antibody in the control cell sample is indicative of the presence of disease in the test cell sample.

### BRIEF DESCRIPTION OF THE FIGURES

FIG. 1A illustrates the expression levels in Parkinson's and Alzheimer's brain tissue and white blood cells of a leukemia patient of the GPCR gi6863021_GS_nt7 having the polynucleotide sequence shown in FIG. 1B (SEQ ID NO 1), the amino acid sequence of shown in FIG. 1C (SEQ ID NO 6) and the Kyte-Doolittle hydropathicity plots and “DRY” motif is shown in FIG. 1D.

FIG. 2A illustrates the expression levels in Parkinson's and Alzheimer's brain tissue and white blood cells of a leukemia patient of the GPCR gi6453999_GS_nt6 having the polynucleotide sequence shown in FIG. 2B (SEQ ID NO 2), the amino acid sequence of shown in FIG. 2C (SEQ ID NO 7) and the Kyte-Doolittle hydropathicity plots and “DRY” motif is shown in FIG. 2D.

FIG. 3A illustrates the expression levels in Parkinson's and Alzheimer's brain tissue and white blood cells of a leukemia patient of the GPCR gi6671985_GS_nt9.2 having the polynucleotide sequence shown in FIG. 3B (SEQ ID NO 3), the amino acid sequence of shown in FIG. 3C (SEQ ID NO 8) and the Kyte-Doolittle hydropathicity plots and “DRY” motif is shown in FIG. 3D.

FIG. 4A illustrates the expression levels in Parkinson's and Alzheimer's brain tissue and white blood cells of a leukemia patient of the GPCR gi5791525_GS_nt10 having the polynucleotide sequence shown in FIG. 4B (SEQ ID NO 4), the amino acid sequence of shown in FIG. 4C (SEQ ID NO 9) and the Kyte-Doolittle hydropathicity plots and “DRY” motif is shown in FIG. 4D.

FIG. 5A illustrates the expression levels in Parkinson's and Alzheimer's brain tissue and white blood cells of a leukemia patient of the GPCR gi5791525_GS_nt8 having the polynucleotide sequence shown in FIG. 5B (SEQ ID NO 5), the amino acid sequence of shown in FIG. 5C (SEQ ID NO 10) and the Kyte-Doolittle hydropathicity plots and “DRY” motif is shown in FIG. 5D.

### DETAILED DESCRIPTION OF PREFERRED EMBODIMENTS

The present invention provides novel methods and compositions involving GPCR polypeptides and polynucleotides specifically over-expressed or under-expressed in diseased tissues, specifically (and by example) Parkinson's disease, Alzheimer's disease and leukemia patients. The invention can be used in various aspects of genome analysis that finds utility in both basic biological research and medical diagnosis and therapeutics.

1. Definitions

A “polynucleotide” is a polymeric form of nucleotides of any length, either ribonucleotides or deoxyribonucleotides. This term refers only to the primary structure of the molecule. Thus, this term includes double- and single-stranded DNA and RNA. It also includes known types of modifications, for example, labels which are known in the art, methylation, “caps”, substitution of one or more of the naturally occurring nucleotides with an analog, internucleotide modifications such as, for example, those with uncharged linkages (e.g., phosphorothioates, phosphorodithioates, etc.), those containing pendant moieties, such as, for example proteins (including for e.g., nucleases, toxins, antibodies, signal peptides, poly-L-lysine, etc.),those with intercalators (e.g., acridine, psoralen, etc.), those containing chelators (e.g., metals, radioactive metals, etc.), those containing alkylators, those with modified linkages (e.g., alpha anomeric nucleic acids, etc.), as well as unmodified forms of the polynucleotide.

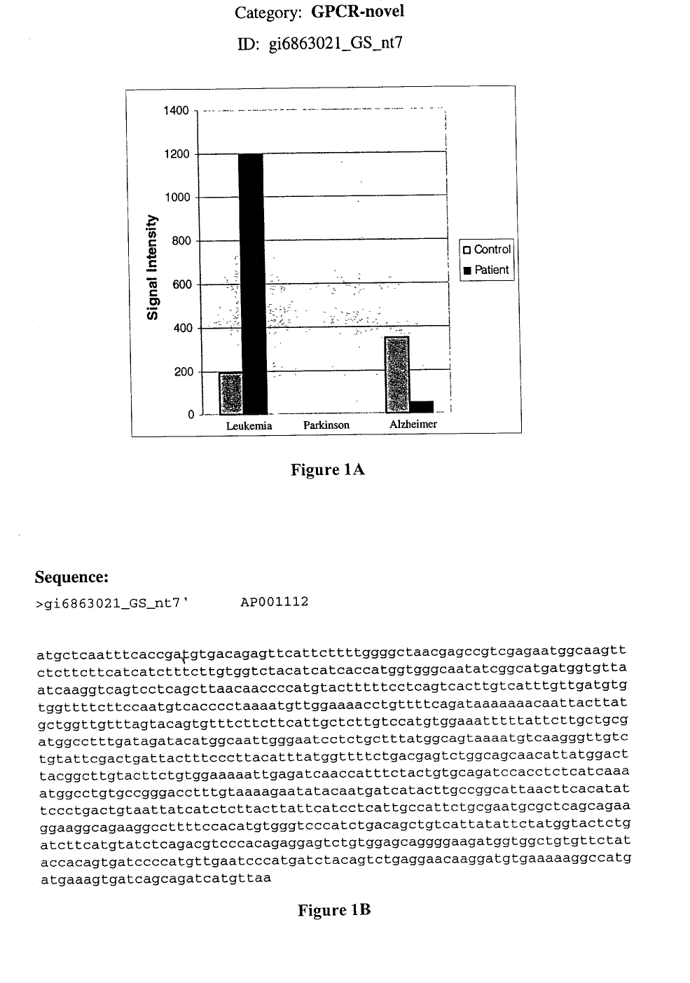

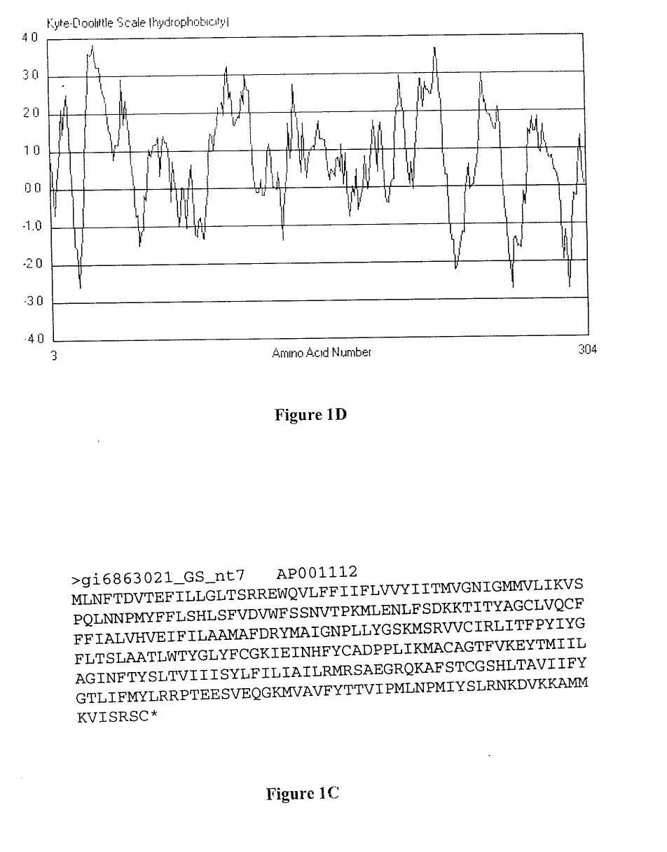

The scope of the invention with respect to polynucleotide compositions useful in the methods described herein includes, but is not necessarily limited to, polynucleotides having a sequence set forth in any one of the polynucleotide sequences FIGS. 1B, 2B, 3B, 4B and 5B (SEQ ID NOS 1-5) provided herein or other biological sources (particularly human sources) or by hybridization to the above mentioned sequences under stringent conditions (particularly conditions of high stringency); genes corresponding to the provided polynucleotides; variants of the provided polynucleotides and their corresponding genes particularly those variants that retain a biological activity of the encoded gene product (e.g., a biological activity ascribed to a gene product corresponding to the provided polynucleotides as a result of the assignment of the gene product to a protein family(ies) and/or identification of a functional domain present in the gene product). Other polynucleotide compositions contemplated by and within the scope of the present invention will be readily apparent to one of ordinary skill in the art when provided with the disclosure here.

“Polynucleotide” and “nucleic acid” are used herein inter-changably with reference to nucleic acids of the composition and is not intended to be limiting as to the length or structure of the nucleic acid unless specifically indicted. As used herein, “polynucleotide” means a polymeric form of nucleotides of any length equal to or longer than a dimer of nucleotides, which contain deoxyribonucleotides, ribonucleotides, and/or their analogs. The terms “polynucleotide”, “oligonucleotide” and “nucleotide” as used herein are used interchangeably. Polynucleotides may have any three-dimensional structure, and may perform any function, known or unknown. The term “polynucleotide” includes double- or single-stranded molecules. Unless otherwise specified or required, any embodiment of the invention described herein that includes a polynucleotide encompasses both the double-stranded form and each of two complementary single-stranded forms known or predicted to make up the double stranded form. The following are non-limiting examples of polynucleotides: a gene or gene fragment, exons, introns, MRNA, tRNA, rRNA, ribozymes, codon, recombinant polynucleotides, branched polynucleotides, plasmids, vectors, any isolated DNA from any source, any isolated RNA from any sequence, nucleic acid probes, and primers.

A polynucleotide or polynucleotide region has a certain percentage (for example, 80%, 85%, 90%, or 95%) of “sequence identity” to another sequence means that, when aligned, that percentage of bases are the same in comparing the two sequences. This alignment and the percent homology or sequence identity can be determined using software programs known in the art, for example those described in Current Protocols in Molecular Biology (F. M. Ausubel et al., eds., 1987) Supplement 30, section 7.7.18. A preferred alignment program is GCG Gap (Genetics Computer Group, Wisconsin, Suite Version 10.1), preferably using default parameters, such as: open gap=3; extend gap=1.

Polynucleotides contemplated by the invention also include naturally occurring variants of the nucleotide sequences (e.g., degenerate variants, allelic variants, etc.). Variants of the polynucleotides contemplated by the invention are identified by hybridization of putative variants with the polynucleotide sequences disclosed herein, preferably by hybridization under stringent conditions. For example, by using appropriate wash conditions, variants of the polynucleotides described herein can be identified where the allelic variant exhibits at most about 25-30% base pair (bp) mismatches relative to the selected polynucleotide probe. In general, allelic variants contain 15-25% bp mismatches, and can contain as little as even 5-15%, or 2-5%, or 1-2% bp mismatches, as well as a single bp mismatch.

The invention also encompasses homologues corresponding to any one of the polynucleotide sequences provided herein, where the source of homologous genes can be any mammalian species, e.g., primate species, particularly human; rodents, such as rats, etc. Between mammalian species, e.g., human and primate, homologues generally have substantial sequence similarity, e.g., at least 75% sequence identity, usually at least 90%, more usually at least 95% between nucleotide sequences. Sequence similarity is calculated based on a reference sequence, which may be a subset of a larger sequence, such as a conserved motif, coding region, flanking region, domain, etc. A reference sequence will usually be at least about 18 contiguous nucleotides long, more usually at least about 30 nucleotides long, and may extend to the complete sequence that is being compared. Algorithms for sequence analysis are known in the art.

The subject nucleic acids can be cDNAs or genomic DNAs, as well as fragments thereof, particularly fragments that encode a biologically active gene product and/or are useful in the methods disclosed herein (e.g., in diagnosis, as a unique identifier of a differentially expressed gene of interest, etc.). The term “codon” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide. mRNA species can also exist with both exons and introns, where the introns may be removed by alternative splicing. Furthermore it should be noted that different species of mRNAs encoded by the same genomic sequence can exist at varying levels in a cell, and detection of these various levels of mRNA species can be indicative of differential expression of the encoded gene product in the cell.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ and 3′ end of the transcribed region. The genomic DNA can be isolated as a fragment of 100 kbp or smaller; and substantially free of flanking chromosomal sequence. The genomic DNA flanking the coding region, either 3′ and 5′, or internal regulatory sequences as sometimes found in introns, contains sequences required for proper tissue, stage-specific, or disease-state specific expression.

As used herein, the term “vector” refers to a polynucleotide construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors”, which comprise the attributes of more than one type of vector.

A “host cell” includes an individual cell or cell culture which can be or has been a recipient of exogenous polynucleotides. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a polynucleotide of this invention.

“Expression” includes transcription and/or translation.

The present invention further provides polypeptides, such as those comprising sequences shown in FIGS. 1C, 2C, 3C, 4C and 5C (SEQ ID NOS 6-10) and those encoded by polynucleotides shown in FIGS. 1B, 2B, 3B, 4B and 5B (SEQ ID NOS 1-5) that are differentially expressed in tissue samples. The polypeptides can be used to generate antibodies specific for a polypeptide associated with Parkinson's disease, Alzheimer's disease and leukemia, which antibodies are in turn useful in diagnostic methods, prognostic methods, therametric methods, and the like as discussed in more detail herein. Polypeptides are also useful as targets for therapeutic intervention, as discussed in more detail herein. The invention includes within its scope a polypeptide encoded by a polynucleotide having the sequence of any one of the polynucleotide sequences provided herein, or a variant thereof. The polypeptides of the present invention encompass polypeptides made by any method including isolation from a tissue or cell line source, recombinant expression, or chemical synthesis.

In general, the term “polypeptide” as used herein refers to both the full length polypeptide encoded by the recited polynucleotide, the polypeptide encoded by the gene represented by the recited polynucleotide, as well as portions or fragments thereof. The present invention encompasses variants of the naturally occurring proteins, wherein such variants are homologous or substantially similar to the naturally occurring protein, and can be of an origin of the same or different species as the naturally occurring protein (e.g., human, murine, or some other species that naturally expresses the recited polypeptide, usually a mammalian species). In general, variant polypeptides have a sequence that has at least about 80%, usually at least about 90%, and more usually at least about 99% sequence identity with a differentially expressed polypeptide described herein, as determined by the Smith-Waterman homology search algorithm using an affine gap search with a gap open penalty of 12 and a gap extension penalty of 2, BLOSUM matrix of 62. The Smith-Waterman homology search algorithm is taught in Smith and Waterman, Adv. Appl. Math. (1981) 2: 482-489.. The variant polypeptides can be naturally or non-naturally glycosylated, i.e., the polypeptide has a glycosylation pattern that differs from the glycosylation pattern found in the corresponding naturally occurring protein.

The invention also encompasses homologues of the disclosed polypeptides (or fragments thereof) where the homologues are isolated from other mammalian species, e.g. rodents, such as mice, rats; and primates and humans. By “homologue” is meant a polypeptide having at least about 35%, usually at least about 40% and more usually at least about 60% amino acid sequence identity to a particular differentially expressed protein as identified above, where sequence identity is determined by the Smith-Waterman homology search algorithm using an affine gap search with a gap open penalty of 12 and a gap extension penalty of 2, BLOSUM matrix of 62. The Smith-Waterman homology search algorithm is taught in Smith and Waterman, Adv. Appl. Math. (1981) 2: 482-489. In general, the polypeptides of the subject invention are provided in a non-naturally occurring environment, e.g. are separated from their naturally occurring environment. In certain embodiments, the subject protein is present in a composition that is enriched for the protein as compared to a control. As such, purified polypeptide is provided, where by purified is meant that the protein is present in a composition that is substantially free of other expressed polypeptides, where by substantially free is meant that less than 90%, usually less than 60% and more usually less than 50% of the composition is made up of other expressed polypeptides.

Also within the scope of the invention are variants. Variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. Variants can be designed so as to retain or have enhanced biological activity of a particular region of the protein (e.g., a functional domain and/or, where the polypeptide is a member of a protein family, a region associated with a consensus sequence). Selection of amino acid alterations for production of variants can be based upon the accessibility (interior vs. exterior) of the amino acid (see, e.g., Go et al, Int. J Peptide Protein Res. (1980) 15:211), the thermostability of the variant polypeptide (see, e.g., Querol et al., Prot. Eng. (1996) 9:265), desired glycosylation sites (see, e.g., Olsen and Thomsen, J. Gen. Microbiol. (1991) 137:579), desired disulfide bridges (see, e.g., Clarke et al., Biochemistry (1993) 32:4322; and Wakarchuk et al., Protein Eng. (1994) 7:1379), desired metal binding sites (see, e.g., Toma et al., Biochemistry (1991) 30:97, and Haezerbrouck et al., Protein Eng. (1993) 6:643), and desired substitutions with in proline loops (see, e.g., Masul et al., Appl. Env. Microbiol. (1994) 60:3579). Cysteine-depleted muteins can be produced as disclosed in U.S. Pat. No. 4,959,314.

Variants also include fragments of the polypeptides disclosed herein, particularly biologically active fragments and/or fragments corresponding to functional domains. Fragments of interest will typically be at least about 8 amino acids (aa) 10 aa, 15 aa, 20 aa, 25 aa, 30 aa, 35 aa, 40 aa, to at least about 45 aa in length, usually at least about 50 aa in length, at least about 75 aa, at least about 100 aa, at least about 125 aa, at least about 150 aa in length, at least about 200 aa, at least about 300 aa, at least about 400 aa and can be as long as 500 aa in length or longer, but will usually not exceed about 1000 aa in length, where the fragment will have a stretch of amino acids that is identical to a polypeptide encoded by a polynucleotide having a sequence of any one of the polynucleotide sequences provided herein, or a homologue thereof. The protein variants described herein are encoded by polynucleotides that are within the scope of the invention. The genetic code can be used to select the appropriate codons to construct the corresponding variants.

While the over-expression of the polynucleotides associated with Parkinson's disease, Alzheimer's disease and leukemia is observed, elevated levels of expression of the polypeptides encoded by these polynucleotides may likely play a role in Parkinson's disease, Alzheimer's disease and leukemia.

A “G-protein” or “GTP-binding protein” is one of a superfamily of proteins that function in, for example, signal transduction (e.g. the G-protein associated with the b-adrenergic receptor), polymerization (e.g. tubulin), ribosomal protein synthesis (e.g. the translocase), cell differentiation (ras proteins) and intracellular transport of proteins, vesicles or cytoskeletal elements (e.g. dynamin). The common functional feature of the family is an affinity for a target protein in the presence of GTP which is lost upon hydrolysis to GDP. (see, Boume, H. R., Sanders, D. A. and McCormick, F. (1990) Nature (London) 348, 125-132; Hilgenfeld, R. (1995) Curr. Opin. Struct. Biol. 5, 810-817).

The class of receptor termed “G-protein coupled receptors (GPCRs)” couple signal transduction to G-proteins. GPCRs are coupled, inside the cell, to GTP-binding and hydrolyzing proteins (termed G-proteins). Receptors of the class that interact with G-proteins all have a structure that is characterized by 7 transmembrane (7-TM) spanning domains. These receptors are also termed serpentine receptors. Examples of this class are the adrenergic receptors, odorant receptors, and certain hormone receptors (e.g. glucagon, angiotensin, vasopressin and bradykinin).

A “microarray” is a linear or two-dimensional array of preferably discrete regions, each having a defined area, formed on the surface of a solid support. The density of the discrete regions on a microarray is determined by the total numbers of target polynucleotides to be detected on the surface of a single solid phase support, preferably at least about 50/cm², more preferably at least about 100/cm², even more preferably at least about 500/cm², and still more preferably at least about 1,000/cm². As used herein, a DNA microarray is an array of oligonucleotide primers placed on a chip or other surfaces used to amplify or clone target polynucleotides. Since the position of each particular group of primers in the array is known, the identities of the target polynucleotides can be determined based on their binding to a particular position in the microarray.

The term “label” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

As used herein, a “biological sample” refers to a sample of tissue or fluid isolated from an individual, including but not limited to, for example, blood, plasma, serum, spinal fluid, lymph fluid, the external sections of the skin, respiratory, intestinal, and genitourinary tracts, tears, saliva, milk, cells (including but not limited to blood cells), tumors, organs, and also samples of in vitro cell culture constituents.

The term “biological sources” as used herein refers to the sources from which the target polynucleotides are derived from. The source can be of any form of “sample” as described above, including but not limited to, cell, tissue or fluid. “Different biological sources” can refer to different cells/tissues/organs of the same individual, or cells/tissues/organs from different individuals of the same species, or cells/tissues/organs from different species.

2. Methods and Materials

Selecting Oligonucleotide Probes

The invention provides a prepared solid support comprising immobilized and separate groups of oligonucleotide probes. Each probe group corresponds to a particular region within the reference sequence, and contains at least four sets of probes with the first set being exactly complementary to the particular region of the reference sequence, and the other three sets being identical to the first set but for the most 3′-end nucleotide. For example, for an A nucleotide in the reference sequence, the corresponding probe from the first set has a T at its most 3′-end, while the additional three probe sets have at their most 3′-end an A, C, or G, a different nucleotide in each set. The length of the four probe sets are preferred, although not necessary, to be the same. The probes can be selected or designed using for example a standard PCR probe selection program such as Probe3 from Massachusetts Institute of Technology (MIT).

The solid phase support can provide an areas of about 5 to about 100 square micrometers, on which up to about 100,000 groups of probes can be immobilized in discrete areas according to a predetermined pattern. The prepared solid support can have an associated written or electronic record of the sequence of the probe or probe pairs at any given location on the support, and thus the location on the support of an amplified target can be identified as well.

The number of probes within each group corresponding to a particular region of the reference sequence can be determined and limited by the needs of the subsequent planned amplification reaction on the microarray. Thus, for example, the number of probes deemed necessary for conducting an PCR at a specific site on the microarray, given especially the reaction volume and expected number of target template polynucleotide molecules, and the proposed number of cycles of PCR, will help determine exactly how much oligonucleotide probe copies to apply as a group at each location on the support to ensure successful reactions. Preferably, the amounts of probes (i.e. probe molecule numbers or probe concentration) will be about the same at each provided location on a given solid support (e.g. in a DNA microarray format having from 1000, to 10,000, up to about 100,000 groups of probes to amplify or detect up to about 100,000 regions of the target polynucleotide).

The solid support can be prepared with probe sequences for a particular application based on the polynucleotides to be detected. The oligonucleotide probes can be of any length suitable for a particular PCR, especially considering the sequence and quality of the target polynucleotides to be amplified. As an example, the probes can be from about 4 to about 30 nucleotides in length.

It is understood that a nucleic acid probe of the present invention may contain minor deletions, additions and/or substitutions of nucleic acid bases, to the extent that such alterations do not negatively affect the yield or product obtained to a significant degree.

Oligonucleotide probes can include the naturally-occurring heterocyclic bases normally found in nucleic acids (uracil, cytosine, thymine, adenine and guanine), as well as modified bases and base analogues. Any modified base or base analogue compatible with hybridization of the probe to a target sequence is useful in the practice of the invention.

The sugar or glycoside portion of the probe can comprise deoxyribose, ribose, and/or modified forms of these sugars, such as, for example, 2′-O-alkyl ribose. In a preferred embodiment, the sugar moiety is 2′-deoxyribose; however, any sugar moiety that is compatible with the ability of the probe to hybridize to a target sequence can be used.

In one embodiment, the nucleoside units of the probe are linked by a phosphodiester backbone, as is well known in the art. In additional embodiments, internucleotide linkages can include any linkage known to one of skill in the art that is compatible with specific hybridization of the probe including, but not limited to phosphorothioate, methylphosphonate, sulfamate (e.g., U.S. Pat. No. 5,470,967) and polyamide (i.e., peptide nucleic acids). Peptide nucleic acids are described in Nielsen et al. (1991) Science 254: 1497-1500, U.S. Pat. No. 5,714,331, and Nielsen (1999) Curr. Opin. Biotechnol. 10:71-75.

In certain embodiments, the probe can be a chimeric molecule; i.e., can comprise more than one type of base or sugar subunit, and/or the linkages can be of more than one type within the same probe.

The probe can comprise a moiety to facilitate hybridization to its target sequence, as are known in the art, for example, intercalators and/or minor groove binders.

Variations of the bases, sugars, and internucleoside backbone, as well as the presence of any pendant group on the probe, will be compatible with the ability of the probe to bind, in a sequence-specific fashion, with its target sequence. A large number of structural modifications, both known and to be developed, are possible within these bounds. Moreover, synthetic methods for preparing the various heterocyclic bases, sugars, nucleosides and nucleotides which form the probe, and preparation of oligonucleotides of specific predetermined sequence, are well-developed and known in the art. A preferred method for oligonucleotide synthesis incorporates the teaching of U.S. Pat. No. 5,419,966.

The oligonucleotide probes can be designed with any special additional moieties or sequences that will aid and facilitate a particular PCR or subsequent manipulations, e.g. isolation of the amplified target polynucleotides. For example, a probe can comprise sequences in addition to those that are complementary to the target sequence. Such sequences are normally upstream (i.e., to the 5′-side) of the target-complementary sequences in the probe. For example, sequences comprising one or more restriction enzyme recognition sites (so-called “linkers” or “adapters”), when present in a probe upstream of target-complementary sequences, facilitate cloning and subsequent manipulation of an amplification product. Other useful sequences for inclusion in a probe include those complementary to a sequencing probe and those specifying a promoter for a bacteriophage RNA polymerase, such as, for example, T3 RNA polymerase, T7 RNA polymerase and/or SP6 RNA polymerase.

In one aspect of the invention, the microarray probes are defined by a tiling method to cover an entire region of interest in the target polynucleotide. For example, a first group of probes are designed so that the sequence of each probe therein corresponds to the most 5′-portion of the region of interest; a second group of probes have sequence that is “shifted” from the first group by one nucleotide towards the 3′-end of the region; and a third group of probes have sequence that is “shifted” from the second group by one nucleotide toward the 3′-end of the region, and etc. In theory, then, the number of groups of probes equals to the number of nucleotides in the region of interest. Of course, within each group of probes that correspond to a particular portion of the region, there are at least four sets of probes with four different 3′-ends as described above. When multiple target polynucleotides are to be detected according to the present invention, each probe group corresponding to a particular target polynucleotide is resided in a discrete area of the microarray.

Solid Phase Supports

5′-modified oligonucleotides representing novel GPCR sequences and optimized for DNA microarray analysis are spotted on DNA microarray slides. The microarray slides of the present invention can be of any solid materials and structures suitable for supporting nucleotide hybridization and synthesis. Preferably, the solid phase support comprises at least one substantially rigid surface on which the primers can be immobilized and the PCR reaction performed. The solid phase support can be made of, for example, glass, synthetic polymer, plastic, hard non-mesh nylon or ceramic. Other suitable solid support materials are known and readily available to those of skill in the art. The size of the solid support can be any of the standard microarray sizes, useful for DNA microarray technology, and the size may be tailored to fit the particular machine being used to conduct a reaction of the invention. Methods and materials for derivatization of solid phase supports for the purpose of immobilizing oligonucleotides are known to those skill in the art and described in, for example, U.S. Pat. No. 5,919,523, the disclosure of which is incorporated herein by reference.

Probe Immobilization

The oligonucleotide primers of the invention are affixed, immobilized, provided, and/or applied to the surface of the solid support using any available means to fix, immobilize, provide and/or apply the oligonucleotides at a particular location on the solid support. For example, photolithography (Affymetrix, Santa Clara, Calif.) can be used to apply the oligonucleotide primers at particular position on a chip or solid support, as described in the U.S. patents, U.S. Pat. Nos. 5,919,523, 5,837,832, 5,831,070, and 5,770,722, which are incorporated herein by reference. The oligonucleotide primers may also be applied to a solid support as described in Brown and Shalon, U.S. Pat. No. 5,807,522 (1998). Additionally, the primers may be applied to a solid support using a robotic system, such as one manufactured by Genetic MicroSystems (Woburn, Mass.), GeneMachines (San Carlos, Calif.) or Cartesian Technologies (Irvine, Calif.).

PCR Amplifications

In practicing the invention, a reaction mixture comprising the appropriate target polynucleotides mixed with the reagents necessary for conducting the polymerase chain reaction (PCR) are placed in contact with each immobilized primer pair or single primer population on the solid support. The appropriate target polynucleotides can be double stranded DNA, single stranded codon generated by reverse transcription of RNA templates, or mRNA population. The reaction mixture contains an enzyme for facilitating the synthesis of a polynucleotide strand complementary to a target strand. Suitable polymerases include thermostable polymerase enzymes, such as Taq DNA polymerase, TthI DNA polymerase, The DNA polymerase, Tma DNA polymerase, Pfu DNA polymerase, Vent DNA polymerase or any other thermostable DNA polymerase. The reaction mixture can also contain a label molecule capable of being incorporated into the nascent strands during polymerase chain reaction so that the amplified products can be detected on the solid support after the PCR. The label can be detected directly or indirectly according to methods well known in the art. Suitable labels for direct detection can be any fluorescent molecules such as fluorescein isothiocyanate, Texas red or rhodamine. Molecules facilitating indirect detection, such as biotin or digoxigenin, can also be incorporated into the nascent strands during the PCR. Biotin can be subsequently detected by binding to a labeled streptavidin or a labeled anti-biotin antibody. Likewise, incorporated digoxigenin can be detected by a labeled or unlabeled anti-digoxigenin antibody, and the unlabeled anti-digoxigenin antibody can be detected by binding a labeled anti-anti-digoxigenin antibody.

Labeling and Detection

Detecting the amplified or labeled target polynucleotides can be conducted by standard methods used to detect the labeled sequences, including for example, detecting labels that have been incorporated into the amplified or newly synthesized DNA strands. Thus, for example fluorescent labels or radiolabels can be detected directly. Other labeling techniques may require that a label such as biotin or digoxigenin that is incorporated into the DNA during strand synthesis be detected by an antibody or other binding molecule (e.g. streptavidin) that is either labeled or which can bind a labeled molecule itself, for example, a labeled molecule can be e.g. an anti-streptavidin antibody or anti-digoxigenin antibody conjugated to either a fluorescent molecule (e.g. fluorescein isothiocyanate, Texas red and rhodamine), or conjugated to an enzymatically activatable molecule. Whatever the label on the newly synthesized molecules, and whether the label is directly in the DNA or conjugated to a molecule that binds the DNA (or binds a molecule that binds the DNA), the labels (e.g. fluorescent, enzymatic, chemiluminescent, or colorimetric) can be detected by a laser scanner or a CCD camera, or X-ray film, depending on the label, or other appropriate means for detecting a particular label.

The target polynucleotides can be detected by using labeled nucleotides (e.g. dNTP-fluorescent label for direct labeling; dNTP-biotin or dNTP-digoxigenin for indirect labeling) are incorporated into amplified DNA during the PCR. For indirectly labeled DNA, the detection is carried out by fluorescence or other enzyme conjugated streptavidin or anti-digoxigenin antibodies. The PCR method employs detection of the polynucleotides by detecting incorporated label in the newly synthesized complements to the polynucleotide targets. For this purpose, any label that can be incorporated into DNA as it is synthesized can be used, e.g. fluoro-dNTP, biotin-dNTP, or digoxigenin-dNTP, as described above and are known in the art. PCR amplification conducted using one or more universal primers in solution provides the option to detect the amplified targets at locations on the solid support by detecting the universal primers. Thus, where more than one universal primer is used, target strands from different sources can be differentially detected on the solid support.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, OR and Research Organics, Cleveland, Ohio.

Labeled nucleotides are preferred form of detection label since they can be directly incorporated during synthesis. Examples of detection labels that can be incorporated into amplified DNA or RNA include nucleotide analogs such as BrdUrd (Hoy and Schimke, Mutation Research 290:217-230 (1993)), BrUTP (Wansick et al., J. Cell Biology 122:283-293 (1993)) and nucleotides modified with biotin (Langer et al., Proc. Natl. Acad. Sci. USA 78:6633 (1981)) or with suitable haptens such as digoxigenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (BUDR triphosphate, Sigma), and a preferred nucleotide analog detection label for RNA is Biotin-16-uridine-5′-triphosphate (Biotin-16-dUTP, Boehringer Mannheim). Fluorescein, Cy3, and Cy5 can be linked to dUTP for direct labeling. Cy3.5 and Cy7 are available as avidin or anti-digoxigenin conjugates for secondary detection of biotin- or digoxigenin-labeled probes.

Comparing Differential Expression of Target Polynucleotides from Different Biological Sources

For most studies involving gene expression, RNA is isolated from specific tissue samples. This RNA is then subjected to reverse transcription using oligo-dT primers and fluorescently labeled dNTPs (usually Cy3 or Cy5 labeled dCTP) resulting in a DNA probe that is fluorescently labeled and has a complementary sequence to the original MRNA. The next step is to hybridize the probe to the immobilized target DNA attached to the microarray. This is done by denaturing the probe with heat or a mild base to reduce secondary structures that may have formed and applying it onto the microarray. A cover slip is applied to the array to ensure even distribution of the probe. The array is placed in a warm, humidified chamber overnight to allow the single stranded probe DNA to bind to its complementary single stranded target. The microarray is then removed and washed to remove any nonspecifically bound probe. The arrays are then imaged with a confocal laser scanner. The scanner contains 2 lasers tuned to excite the dye incorporated into the DNA probe and a corresponding filter set to select out excitation emission from the dye (Cy3 or Cy5). The ability to image two fluorescent signals allows for two different RNA samples to be hybridized and directly compared on the same array. This excitation emission signal is recorded via a photomultiplier tube (PMT), digitized, and sent to the computer for later analysis. By examining the intensity of a spot's fluorescence, and the ratio of fluorescence between spots, it possible to determine whether a specific gene is being expressed and the relative expression level of the gene between samples. Other available means for labeling and detecting probes, such as with radioisotopes, enzymes, antibodies, biotin, avidin and like materials known in the art, are within the contemplated means of executing the process.

Solid phase amplification methods can be used to detect and compare gene expressions in different biological sources. The immobilized primers are used in combination with solution phase primers to conduct amplification reactions. Different sources can be different tissues or cells of the same subject. Alternatively, different sources can be comparable tissues of two or more different subjects of same species, e.g., one from a healthy control, and another from a patient. In another embodiment, the different sources are two or more different species or different animals, such as one of human and another of mouse.

As an illustration, the original probe polynucleotides from different biological sources are total mRNAs expressed therein. Methods and materials that are known in the art are used to isolate total mRNAs from each source. The total pool of isolated mRNA from each biological source is then used to prepare a batch of specifically tagged codon that are to be used as “probe polynucleotides” for subsequent amplification. Each batch of the reverse-transcribed cDNAs are tagged with a specific sequence tag at their 3-ends. The specific sequence tag is not present in any of the unmodified probe polynucleotides. For example, if the probe polynucleotides to be detected are from human cells/tissues, the sequence tag can be derived from a bacteria or viral genome such that the sequence does not anneal under hybridization/amplification conditions with the human sequences that are transcribed into MRNA. In this way, the sequence tags from one batch will not cause artifactual amplification of another batch due to cross-hybridization.

Furthermore, the sequence tag for each batch of codon probes is different from that for another batch of codon probes so that they can be compared. The sequence tag can be introduced into the reverse-transcribed codon by using a specially designed primer for reverse transcription. For example, a primer can have a poly-dT portion at its 3′-end and a bacterial SP6 sequence at its 5′-end. During reverse transcription, mRNAs serve as template for a codon synthesis initiated from the 5′-end of the poly-dT portion that anneals with the poly-A tail of the MRNA templates. The resulting codon products then have at their 5′-end a SP6 “sequence tag,” which is unique to this batch of cDNAs. Similarly, a different batch of cDNAs from another source can be “tagged” with a different sequence tag, such as a bacterial T7 sequence.

Two batches of cDNAs differentially tagged with, for example, SP6 and T7 are mixed together for amplification and detection. Present in the amplification reaction mixture are differentially labeled, free SP6 and T7 sequence tags. For example, the two sequence tags can be labeled either with two different fluorescent dyes (e.g., one red dye and one green dye) for direct detection or, alternatively, with two chemical moieties (e.g., one biotin and one digoxigenin) for subsequent color detection. It is important that the labels do not occur at the 3′-end of the sequence tags so that the sequence tags can latter serve as primers in amplification reaction.

A preferred amplification means for the invention is PCR reaction. The mixture of two differentially tagged batches of cDNAs are contacted with an array of multiple groups of specific primer targets, with each group corresponding to a particular probe codon as described above. In the initial round of PCR, the immobilized primers anneal with probe polynucleotides from both sources and synthesize a nascent complementary strand under conditions sufficient for chain elongation. The nascent complementary strand spans through the probe sequence region and contains at its 3′-end a sequence complementary to the sequence tag at the end of the probe codon template. For example, a first nascent strand has a 3′-end complementary to the SP6 sequence if it was amplified on a SP6-tagged codon template from source 1; and a second nascent strand has a 3′-end complementary to the T7 sequence if it was amplified on a T7-tagged codon template from source 2. Thus, each nascent strand immobilized on the solid phase array “inherits” the specific sequence tag specific to its source.

In the subsequent rounds of PCR, the first set of nascent strands tagged for different sources serve as templates for the synthesis of a second set of nascent strands. This time, the initial primers of the PCR are the differentially labeled free sequence tags in solution, such as fluorescein-labeled SP6 primers and lissamine-labeled T7 primers. Accordingly, the second set of nascent strands extended from the labeled sequence tags are differentially labeled corresponding to the original sources of the probe codon templates in the original round of PCR reaction. After washing off the unbound reagents and original templates without denaturation, each immobilized primer site will have a double stranded polynucleotide having on one strand a label indicating the original biological source. As such, the final detection of different labels will reveal the presence and abundance of particular probe polynucleotides in different biological sources.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all and only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1

### Generation of Oligonucleotide Probe Arrays

Putative GPCR sequences, based on well-known GPCR characteristics including the 7 trans-membrane (7-TM) structure, the “DRY” (amino acids Asp-Arg-Tyr) motif and homologies to known human GPCR sequences were searched among human genomic sequences in public database GenBank (NCBI). The GENSCAN bioinformatics software (Burge, C. & Karlin, S. Prediction of complete gene structures in human genomic DNA. J. Mol. Biol. 268, 78-94. (1997); v.1999 version under license from Stanford University, CA) and in house filtering software were used for the search.

A semi-automatic protein families database of alignments and hidden Markov models (PFAM) software (Pfam, release 5) was used to analyze the putative GPCR sequences and capture “all human GPCR sequences” from the input. (see generally, Sonnhammer et al. (1997) Protein 28:405-420).

All “known” human GPCR sequences in GenBank were then subtracted from the “all human GPCR” sequences obtained from the Pfam analysis and the remaining sequences were designated as “novel” GPCR sequences. Amino acid sequences corresponding to the “novel” GPCR sequences were then analyzed by a Kyte-Doolittle hydropathicity plot. (Kyte, J. and Doolittle, R. F. “A Simple Method for Displaying the Hydropathic Character of a Protein”, J. Mol. Biol. 157:105-132 (1982)). Kyte-Doolittle hydrophobicity analysis identified the seven hydrophobic domains characteristic of G-protein coupled receptors and “DRY” motif was searched for by scanning the amino acid sequence.

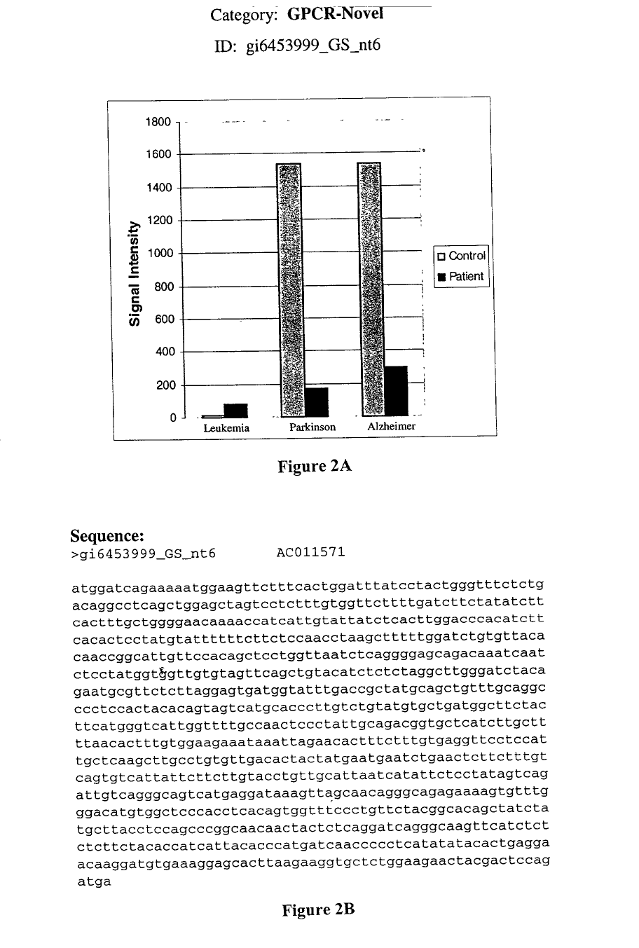

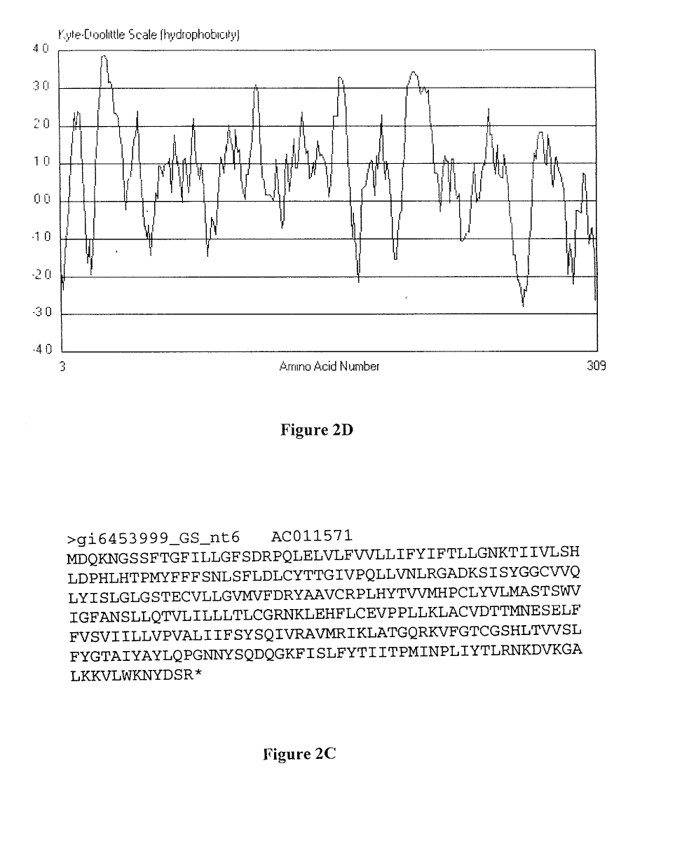

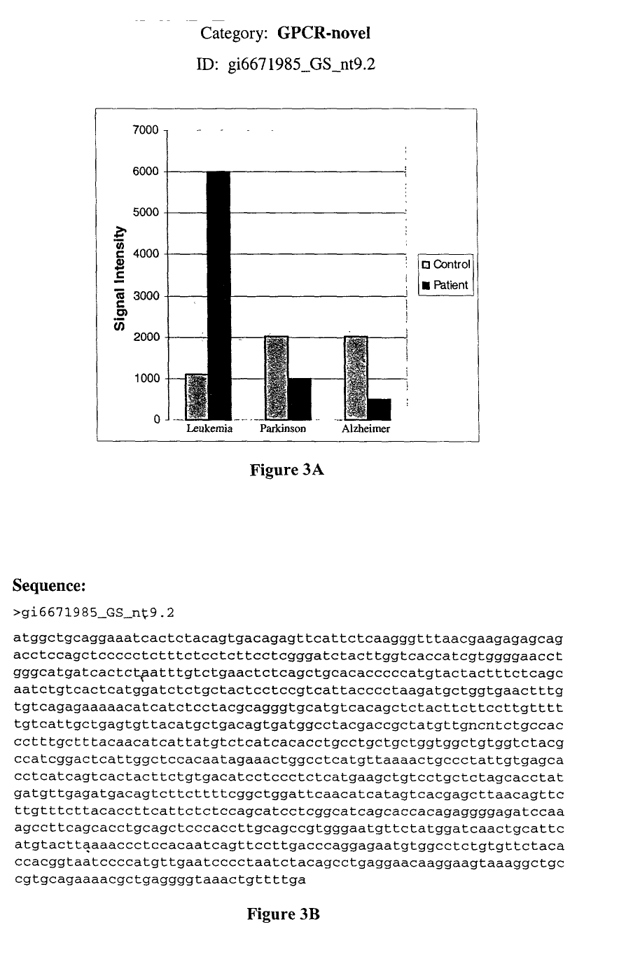

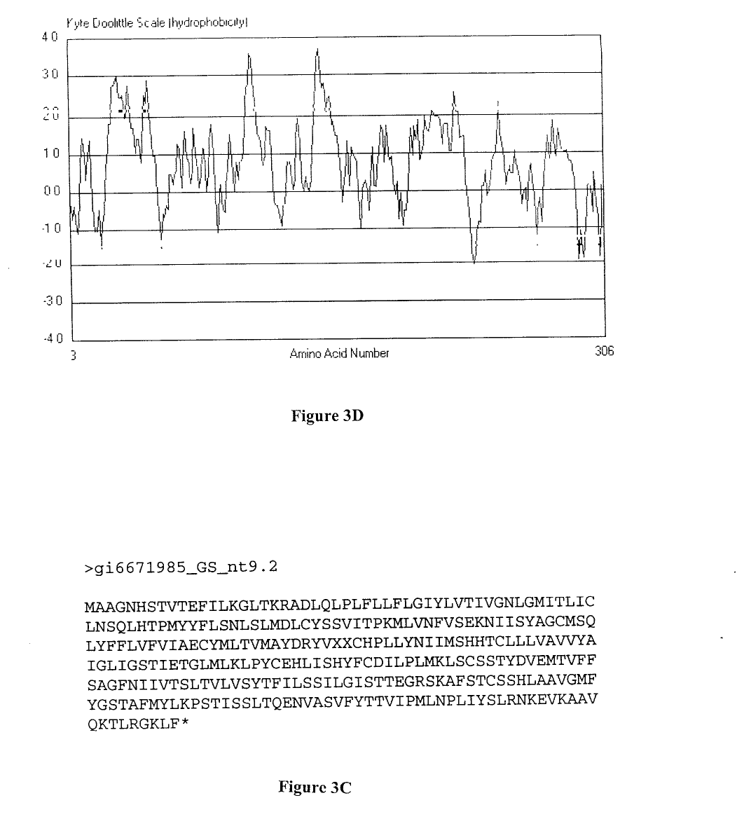

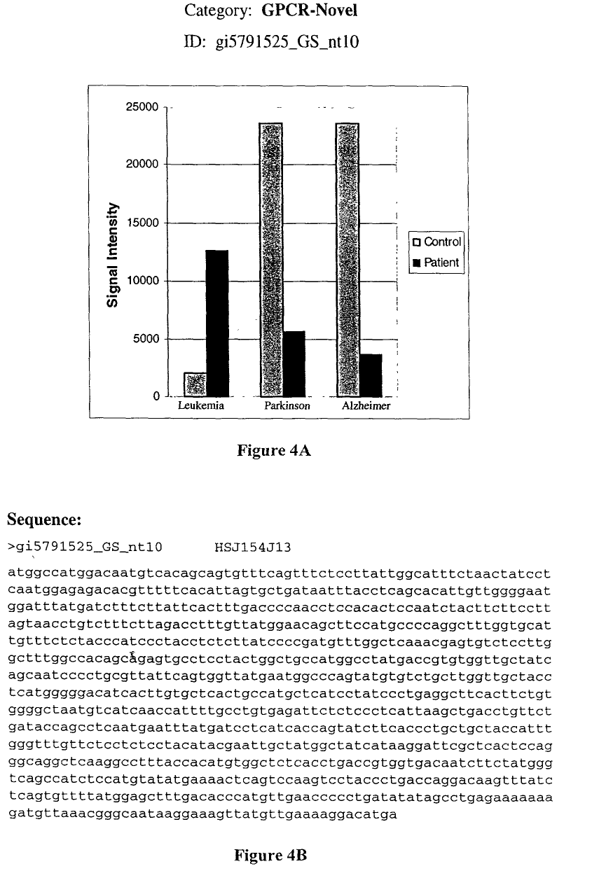

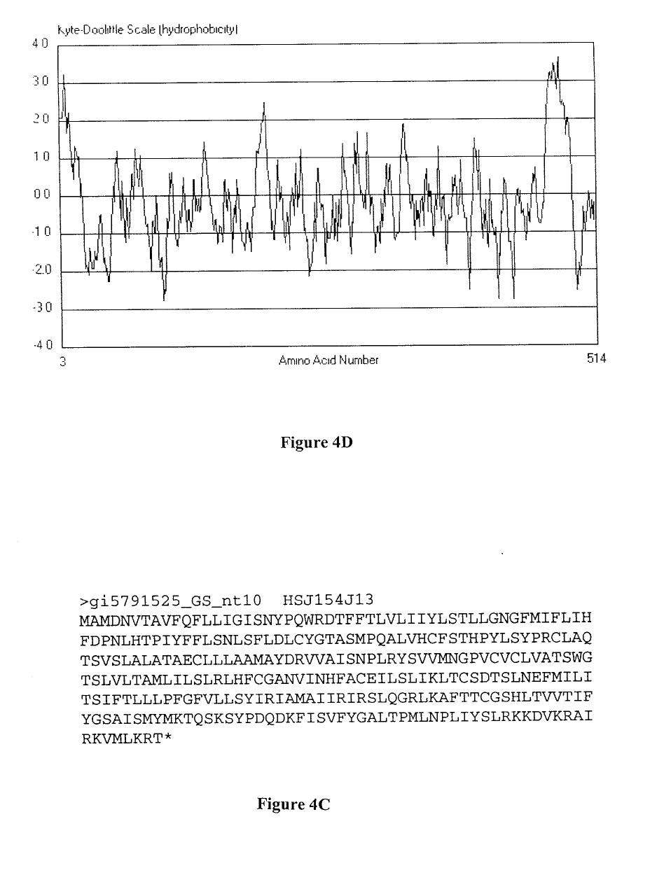

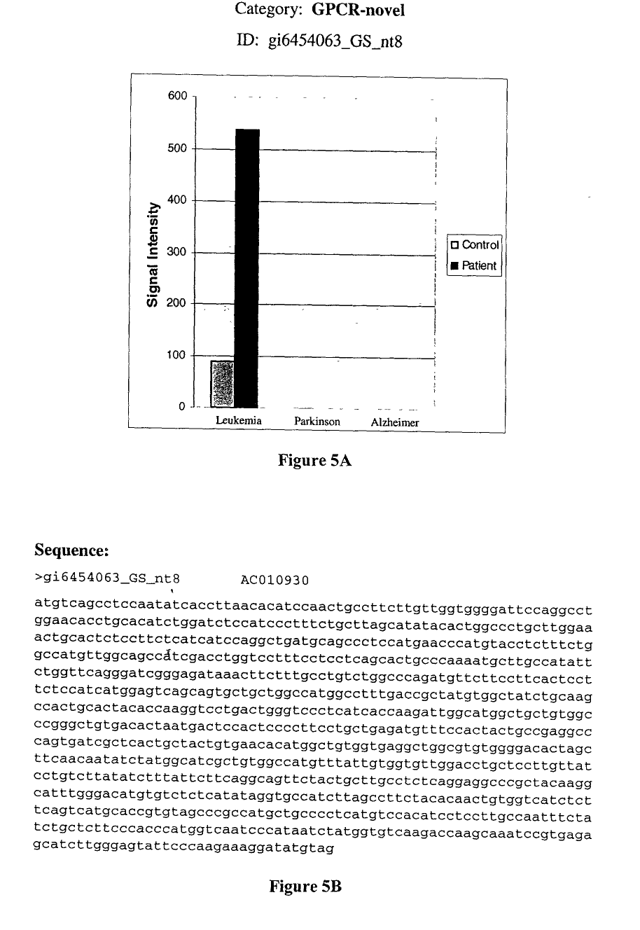

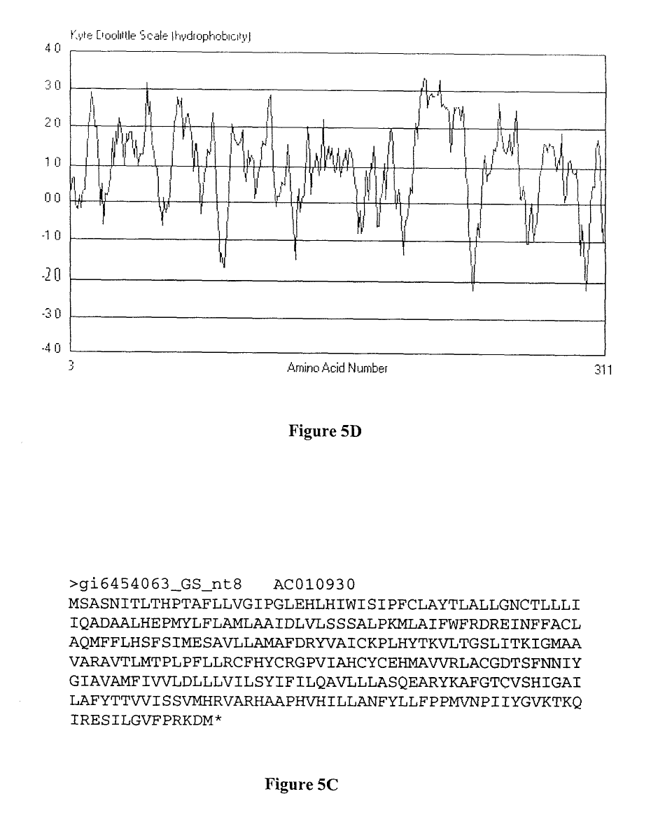

Novel sequences that matched the sequence and structural criteria for GPCR were targeted for full-length clone isolation from genomic and codon libraries. The full-length clones were sequenced, and Kyte-Doolittle hydropathicity plots and “DRY” motif amino acid sequence analyses were subsequently performed to confirm their status in the GPCR family. The amino acid sequence of the GPCR polynucleotide gi6863021_GS_nt7 (FIG. 1B; SEQ ID NO 1) is shown in FIG. 1C (SEQ ID NO 6) and the corresponding Kyte-Doolittle hydropathicity plots and “DRY” motif is shown in FIG. 1D. The amino acid sequence of the GPCR polynucleotide gi6453999_GS_nt6 (FIG. 2B; SEQ ID NO 2) is shown in FIG. 2C (SEQ ID NO 7) and the corresponding Kyte-Doolittle hydropathicity plots and “DRY” motif is shown in FIG. 2D. The amino acid sequence of the GPCR polynucleotide gi6671985_GS_nt9.2 (FIG. 3B; SEQ ID NO 3) is shown in FIG. 3C (SEQ ID NO 8) and the corresponding Kyte-Doolittle hydropathicity plots and “DRY” motif is shown in FIG. 3D. The amino acid sequence of the GPCR polynucleotide gi5791525_GS_nt10 (FIG. 4B; SEQ ID NO 4) is shown in FIG. 4C (SEQ ID NO 9) and the corresponding Kyte-Doolittle hydropathicity plots and “DRY” motif is shown in FIG. 4D. The amino acid sequence of the GPCR polynucleotide gi5791525_GS_nt8 (FIG. 5B; SEQ ID NO 5) is shown in FIG. 5C (SEQ ID NO 10) and the corresponding Kyte-Doolittle hydropathicity plots and “DRY” motif is shown in FIG. 5D.

Summary of Polynucleotides Described Herein:

Table 1 provides a summary of polynucleotides isolated as described above and identified as corresponding to a differentially expressed gene (see Example 2 below). Specifically, Table 1 provides the relative over-expression of a GPCR polynucleotide having a particular clone ID in Parkinson's disease, Alzheimer's disease and leukemia tissue samples.

### Example 2

### Detection of Altered Levels of GPCR RNA Expressed in Disease Tissues Using Arrays

GPCR-related sequences representing a variety of candidate genes to be screened for differential expression in Parkinson's disease, Alzheimer's disease and leukemia were assayed by hybridization on polynucleotide arrays. 5′-modified oligonucleotides representing the novel GPCR sequences and optimized for DNA microarray analysis were designed using a combination of GENSCAN, Pfam, Primer3 (release 0.9 (1998) MIT Whitehead Institute) and in-house informatics programs and were spotted onto reflective glass slides (Amersham) according to methods well known in the art. These microarrays were then used to study differential gene expression in selected cell lines and patient tissues. Normal tissues and tissues from Parkinson's disease, Alzheimer's disease and leukemia patients were processed to generate T7 RNA polymerase transcribed polynucleotides, which were, in turn, assessed for expression in the microarrays.

The microarrays were then used to study differential gene expression in various disease samples and appropriate normal control samples. Target polynucleotides were prepared from total RNA obtained by laser capture microdissection (LCM, Arcturus Engineering Inc., Mountain View, Calif.) of diseased tissue samples and normal tissue samples. Total RNA was first reverse transcribed into codon using a primer containing a T7 RNA polymerase promoter, followed by second strand DNA synthesis. The codon was then transcribed in vitro to produce antisense RNA using the T7 promoter-mediated expression (see, e.g., Luo et al. (1999) Nature Med 5:117-122). In one embodiment, biotin labeled antisense RNA is generated from the RNA isolated from sample and control tissue by standard protocols and hybridized to the immobilized oligonucleotides representing the novel GPCRs.

Hybridization is detected by binding the biotinylated antibody with streptavidin which is in turn bound by a first anti-streptavidin antibody and the signals are amplified using a secondary antibody which binds the anti-streptavidin antibody and is conjugated to the fluorescent label Cy3. The fluorescent label is detected by commercially available array scanners. These indirect immunofluorescence techniques are known in the art.

The array images generated by hybridization were captured by a laser array scanner (e.g., GenePix 4000B by Axon Instruments, Foster City, Calif.) and the data was analyzed using DNA array image analysis software (ImaGene 4.0, BioDiscovery, Inc.) and an in-house microarray data analysis software (Mergen).

In one embodiment, the procedure provides for fluorescent labeling of RNA. Probes are labeled by making fluorescently labeled codon from the RNA starting material. Fluorescently labeled cDNAs prepared from the diseased RNA sample are compared to fluorescently labeled cDNAs prepared from normal cell RNA sample. For example, in one embodiment the codon probes from the normal cells are labeled with Cy3 fluorescent dye (green) and the codon probes prepared from the diseased cells are labeled with Cy5 fluorescent dye (red).

The differential expression assay can be performed by mixing equal amounts of probes from disease cells and normal cells of the same patient. The arrays were prehybridized by incubation for about 2 hrs at 60° C. in 5× SSC/0.2% SDS/1 mM EDTA, and then washed three times in water and twice in isopropanol. Following prehybridization of the array, the probe mixture was then hybridized to the array under conditions of high stringency (overnight at 42° C. in 50% formamide, 5× SSC, and 0.2% SDS). After hybridization, the array was washed at 55° C. three times as follows: (i) first wash in 1× SSC/0.2% SDS; (ii) second wash in 0.1× SSC/0.2% SDS; and (iii) third wash in 0.1× SSC.

The arrays were then scanned for green and/or red fluorescence using a Molecular Dynamics Generation III dual color laser-scanner/detector. The images were processed using the AutoGene Microarray Image Analysis System™ (BioDiscovery, Inc., Los Angeles, Calif.) and the data from each scan set was normalized. In one embodiment, the experiment was repeated, this time labeling the two probes with the opposite color in order to perform the assay in both “color directions.” Each experiment was sometimes repeated with two more slides (one in each color direction). The data from each scan was normalized, and the level fluorescence for each sequence on the array expressed as a ratio of the geometric mean of 8 replicate spots/genes from the four arrays or 4 replicate spots/gene from 2 arrays or some other permutation. Table 1 summarizes the results for gene products differentially expressed in the Parkinson's disease, Alzheimer's disease and leukemia tissue samples relative to normal cells.

As shown in FIGS. 1A, 2A, 3A, 4A and 5A and summarized in Table 1, four of the novel GPCR genes showed differential expression patterns in one or more tissue samples from Parkinson's disease, Alzheimer's disease and leukemia as compared to normal samples.

The GPCR polynucleotide gi6863021_GS_nt7 having the sequence shown in FIG. 1B (SEQ ID NO 1) is expressed at about a 7-fold reduced level in Alzheimer's tissue, and a 6-fold increased level in leukemia as shown in FIG. 1A.

The GPCR polynucleotide gi6453999_GS_nt6 having the sequence shown in FIG. 2B (SEQ ID NO 2) is expressed at about a 8-fold reduced level in Parkinson's tissue, a 5-fold reduced level in Alzheimer's tissue, and a 5-fold increased level in leukemia as shown in FIG. 2A.

The GPCR polynucleotide gi6671985_GS_nt9.2 having the sequence shown in FIG. 3B is expressed (SEQ ID NO 3) at about a 2-fold reduced level in Parkinson's tissue, a 4-fold reduced level in Alzheimer's tissue, and a 6-fold increased level in leukemia as shown in FIG. 3A.

The GPCR polynucleotide gi5791525_GS_nt10 having the sequence shown in FIG. 4B (SEQ ID NO 4) is expressed at about a 5-fold reduced level in Parkinson's tissue, a 6-fold reduced level in Alzheimer's tissue, and a 7-fold increased level in leukemia as shown in FIG. 4A.

The GPCR polynucleotide gi5791525_GS_nt8 having the sequence shown in FIG. 5B (SEQ ID NO 5) is expressed at about a 6-fold increased level in leukemia as shown in FIG. 5A

TABLE 1

| | DRY | | | |
|---|---|---|---|---|
| | motif | | | |
| | at | GPCR Expression in | | |
| | (aa | Diseased over Normal Tissue | | |
| Clone ID No. | residue/ | Parkinson's | Alzheimer's | |
| (SEQ ID NO) | total aa) | Disease | Disease | Leukemia |
| gi6863021_GS_nt7 | 119/308 | N/D | 0.143 × | 6 × |
| (SEQ ID NO 1) | | | normal | normal |
| gi6453999_GS_nt6 | 121/312 | 0.120 × | 0.200 × | 5 × |
| (SEQ ID NO 2) | | normal | normal | normal |
| gi6671985_GS_nt9.2 | 168/356 | 0.500 × | 0.250 × | 6 × |
| (SEQ ID NO 3) | | normal | normal | normal |
| gi5791525_GS_nt10 | 21/309 | 0.208 × | 0.167 × | 7 × |
| (SEQ ID NO 4) | | normal | normal | normal |
| gi5791525_GS_nt8 | 123/314 | N/D | N/D | 6 × |
| (SEQ ID NO 5) | | | | normal |

These genes are thus are possible targets for diagnosis and treatment of Parkinson's disease, Alzheimer's disease and leukemia and are also useful targets for drug development in the areas of CNS diseases.

### Example 3

### Expression of Cloned Polynucleotides in Host Cells

To study the protein products of GPCR codon, restriction fragments from the GPCR codon are cloned into the expression vector pMT2 (Sambrook, et al., Molecular Cloning: A Laboratory Manual, Cold Spring Harbor Laboratory Press pp. 16.17-16.22 (1989)) and transfected into COS cells grown in DMEM supplemented with 10% FCS. Transfections are performed employing calcium phosphate techniques (Sambrook, et al (1989) pp. 16.32-16.40, supra) and cell lysates are prepared forty-eight hours after transfection from both transfected and untransfected COS cells. Lysates are subjected to analysis by immunoblotting using anti-peptide antibody.

In immunoblotting experiments, preparation of cell lysates and electrophoresis are performed according to standard procedures. Protein concentration is determined using BioRad protein assay solutions. After semi-dry electrophoretic transfer to nitro-cellulose, the membranes are blocked in 500 mM NaCl, 20 mM Tris, pH 7.5, 0.05% Tween-20 (TTBS) with 5% dry milk. After washing in TTBS and incubation with secondary antibodies (Amersham), enhanced chemiluminescence (ECL) protocols (Amersham) are performed as described by the manufacturer to facilitate detection.

### Example 4

### Generation of Antibodies Against Polypeptides

Polypeptides, unique to GPCR are synthesized or isolated from bacterial or other (e.g., yeast, baculovirus) expression systems and conjugated to rabbit serum albumin (RSA) with m-maleimido benzoic acid N-hydroxysuccinimide ester (MBS) (Pierce, Rockford, Ill.). Immunization protocols with these peptides are performed according to standard methods. Initially, a pre-bleed of the rabbits is performed prior to immunization. The first immunization includes Freund's complete adjuvant and 500 μg conjugated peptide or 100 μg purified peptide. All subsequent immunizations, performed four weeks after the previous injection, include Freund's incomplete adjuvant with the same amount of protein. Bleeds are conducted seven to ten days after the immunizations.

For affinity purification of the antibodies, the corresponding GPCR polypeptide is conjugated to RSA with MBS, and coupled to CNBr-activated Sepharose (Pharmacia, Uppsala, Sweden). Antiserum is diluted 10-fold in 10 mM Tris-HCl, pH 7.5, and incubated overnight with the affinity matrix. After washing, bound antibodies are eluted from the resin with 100 mM glycine, pH 2.5.

### Example 5

### ELISA Assay for Detecting GPCR-Related Sequences

To test blood samples for antibodies that bind specifically to recombinantly produced GPCR antigens, the following procedure is employed. After the recombinant GPCR-related proteins are purified, the recombinant protein is diluted in PBS to a concentration of 5μg/ml (500 ng/100μl). 100 microliters of the diluted antigen solution is added to each well of a 96-well Immulon 1 plate (Dynatech Laboratories, Chantilly, Va.), and the plate is then incubated for 1 hour at room temperature, or overnight at 4° C., and washed 3 times with 0.05% Tween 20 in PBS. Blocking to reduce nonspecific binding of antibodies is accomplished by adding to each well 200 μl of a 1% solution of bovine serum albumin in PBS/Tween 20 and incubation for 1 hour. After aspiration of the blocking solution, 100 μl of the primary antibody solution (anticoagulated whole blood, plasma, or serum), diluted in the range of 1/16 to 1/2048 in blocking solution, is added and incubated for 1 hour at room temperature or overnight at 4° C. The wells are then washed 3 times, and 100μl of goat anti-human IgG antibody conjugated to horseradish peroxidase (Organon Teknika, Durham, N.C.), diluted 1/500 or 1/1000 in PBS/Tween 20, 100 μl of o-phenylenediamine dihydrochloride (OPD, Sigma) solution is added to each well and incubated for 5-15 minutes. The OPD solution is prepared by dissolving a 5 mg OPD tablet in 50 ml 1% methanol in H₂0 and adding 50 μl 30% H₂O₂ immediately before use. The reaction is stopped by adding 25 l of 4M H₂SO₄ Absorbance are read at 490 nm in a microplate reader (Bio-Rad).

### Example 6

### Preparation of Vaccines

The present invention also relates to a method of stimulating an immune response against cells that express GPCR polypeptides in a patient using GPCR polypeptides of the invention that acts as an antigen produced by or associated with a malignant cell. This aspect of the invention provides a method of stimulating an immune response in a human against cells that express a GPCR polynucleotide and/or polypeptide of the present invention. The method comprises the step of administering to a human an immunogenic amount of a polypeptide comprising: (a) the amino acid sequence of a human GPCR protein or (b) a mutein or variant of a polypeptide comprising the amino acid sequence of a human GPCR protein.

### Example 7

### Generation of Transgenic Animals Expressing Polypeptides as a Means for Testing Therapeutics

GPCR nucleic acids are used to generate genetically modified non-human animals, or site specific gene modifications thereof, in cell lines, for the study of function or regulation of disease-related genes, or to create animal models of diseases, including Parkinson's disease, Alzheimer's disease and leukemia. The term “transgenic” is intended to encompass genetically modified animals having an exogenous GPCR gene(s) that is stably transmitted in the host cells where the gene(s) may be altered in sequence to produce a modified protein, or having an exogenous GPCR LTR promoter operably linked to a reporter gene. Transgenic animals may be made through a nucleic acid construct randomly integrated into the genome. Vectors for stable integration include plasmids, retroviruses and other animal viruses, YACs, and the like. Of interest are transgenic mammals, e.g. cows, pigs, goats, horses, etc., and particularly rodents, e.g. rats, mice, etc.

The modified cells or animals are useful in the study of GPCR gene function and regulation. For example, a series of small deletions and/or substitutions may be made in the GPCR genes to determine the role of different domains in Parkinson's disease, Alzheimer's disease and leukemia. Specific constructs of interest include, but are not limited to, anti-sense constructs to block GPCR gene expression, expression of dominant negative GPCR gene mutations, and differential expression of a GPCR gene. Expression of a GPCR gene or variants thereof in cells or tissues where it is not normally expressed or at abnormal times of development is provided. In addition, by providing expression of proteins derived from GPCR in cells in which it is otherwise not normally produced or produced at inadequate levels, changes in cellular behavior can be induced.

DNA constructs for random integration need not include regions of homology to mediate recombination. Conveniently, markers for positive and negative selection are included. For various techniques for transfecting mammalian cells, see Keown et al., Methods in Enzymology 185:527-537 (1990).

For embryonic stem (ES) cells, an ES cell line is employed, or embryonic cells is obtained freshly from a host, e.g. mouse, rat, guinea pig, etc. Such cells are grown on an appropriate fibroblast-feeder layer or grown in the presence of appropriate growth factors, such as leukemia inhibiting factor (LIF). When ES cells are transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting chimeric animals screened for cells bearing the construct. By providing for a different phenotype of the blastocyst and the ES cells, chimeric progeny can be readily detected.

The chimeric animals are screened for the presence of the modified gene and males and females having the modification are mated to produce homozygous progeny. If the gene alterations cause lethality at some point in development, tissues or organs are maintained as allogeneic or congenic grafts or transplants, or in in vitro culture. The transgenic animals may be any non-human mammal, such as laboratory animals, domestic animals, etc. The transgenic animals are used in functional studies, drug screening, etc., e.g. to determine the effect of a candidate drug on Parkinson's disease, Alzheimer's disease and leukemia, to test potential therapeutics or treatment regimens, etc.

All publications and patent applications mentioned in this specification are incorporated herein by reference to the same extent as if each individual publication or patent application were specifically and individually indicated to be incorporated by reference.

The foregoing description of preferred embodiments of the invention has been presented by way of illustration and example for purposes of clarity and understanding. It is not intended to be exhaustive or to limit the invention to the precise forms disclosed. It will be readily apparent to those of ordinary skill in the art in light of the teachings of this invention that many changes and modifications may be made thereto without departing from the spirit of the invention. It is intended that the scope of the invention be defined by the appended claims and their equivalents.

We claim:

1. A method for identifying a gene whose expression level is associated with a disease state, the method comprising:
identifying at least one gene having a nucleic acid sequence encoding a protein comprising a physical characteristic;
selecting a polynucleotide sequence from the nucleic acid sequence, wherein the polynucleotide sequence is specific for a protein comprising the physical characteristic;
detecting a level of expression of the polynucleotide sequence or a complement thereof in a diseased tissue sample;
detecting a level of expression of the polynucleotide sequence or a complement thereof in a normal tissue sample; and
comparing the level of expression of the polynucleotide sequence or a complement in the diseased tissue sample to a level of expression of the gene in the control tissue sample, wherein an altered level of expression of the polynucleotide sequence or a complement in the diseased tissue sample correlates with the disease state.

2. The method of claim 1, wherein the physical characteristic comprises seven transmembrane (7-TM) domains.

3. The method of claim 2, wherein the gene is a novel G-protein linked cell receptor type gene.

4. The method of claim 1, wherein the physical characteristic comprises an amino acid sequence comprising an Asp-Arg-Tyr (DRY) motif.

5. The method of claim 4, wherein the gene is a novel G-protein linked cell receptor type gene.

6. The method of claim 1, wherein the physical characteristic comprises a signal peptide sequence characteristic of a secreted protein.

7. The method of claim 1, wherein the physical characteristic comprises a signal peptide sequence characteristic of a mitochondrial protein.

8. The method of claim 1, wherein the physical characteristic comprises an amino acid sequence characteristic of a structural feature of the protein.

9. The method of claim 1, wherein the physical characteristic comprises an amino acid sequence characteristic of a function of the protein.

10. The method of claim 1, wherein the identification of the gene comprises searching a nucleic acid sequence database for nucleic acid sequences which encode a protein comprising the physical characteristic.

11. The method of claim 10, wherein the nucleic acid sequence database is an electronic library.

12. The method of claim 10, wherein the gene is identified using a search algorithm.

13. The method of claim 1, wherein the identification of the gene comprises selecting at least one gene whose expression is known to correlate with a disease state.

14. The method of claim 1, wherein the detection of the level of expression of the polynucleotide sequence comprises:
selecting at least one isolated oligonucleotide comprising the polynucleotide sequence or a fragment thereof;
contacting the oligonucleotide with a nucleic acid preparation from the tissue sample; and
detecting a level of expression of the polynucleotide sequence by detecting an amount of hybridization of the nucleic acid preparation to the oligonucleotide under stringent conditions.

15. The method of claim 14, wherein the oligonucleotide is attached to a solid support.

16. The method of claim 15, wherein the solid support is a microarray.

17. The method of claim 14, wherein the selection of the polynucleotide sequence comprises determining at least one of a set of factors comprising (i) a redundancy of the sequence, (ii) an efficiency of hybridization to a complementary sequence, and (iii) a likelihood of the polynucleotide sequence comprising an intron.

18. The method of claim 14, wherein the nucleic acid preparation from the tissue sample comprises a detectable label.

19. The method of claim 18, wherein the detectable label is selected from the group consisting of a fluorescent label, an enzymatic label, a chemiluminescent label, a colorimetric label, and a radioactive label.

20. The method of claim 14, wherein the nucleic acid preparation from the tissue sample is amplified before detection.

21. The method of claim 20, wherein the amplification is a conducted by a polymerase chain reaction (PCR).

22. The method of claim 20, wherein the amplification is a conducted by a quantitative polymerase chain reaction (QPCR).

23. The method of claim 1, wherein comparing the level of expression of the gene comprises:
providing at least one isolated oligonucleotide comprising the polynucleotide sequence or a fragment thereof;
contacting the oligonucleotide with an amount of nucleic acid preparation from a disease tissue sample;
contacting the oligonucleotide with an equal amount of nucleic acid preparation from a normal tissue sample; and
comparing the level of expression of the polynucleotide sequence in the tissue samples by detecting an amount of hybridization of each nucleic acid preparation to the oligonucleotide under stringent conditions.

24. The method of claim 23, wherein the polynucleotide is attached to a solid support.

25. The method of claim 24, wherein the solid support is a microarray.

26. The method of claim 23, wherein the nucleic acid preparation is an RNA preparation.

27. The method of claim 26, wherein the RNA preparation is further processed to generate a labeled nucleic acid probe.

28. The method of claim 27, wherein the labeled nucleic acid probe comprises a label coupled to the probe, wherein the label is selected from the group consisting of a biotin, an avidin, a streptavidin, an antibody, an antigen, a peptide, a fluorescent label, an enzymatic label, a chemiluminescent label, a colorimetric label, and a radioactive label.

29. A method for detecting an expression of a gene identified by the method of claim 1 in a cell, the method comprising:
cloning a polynucleotide fragment comprising a sequence of the cloned gene in an expression vector; and
detecting a corresponding protein in a cell transformed with the vector comprising the cloned fragment.

30. The method of claim 29, wherein the protein is detected by an antibody.

31. The method of claim 29, wherein the protein is detected by a monoclonal antibody.

32. A method for preparing an antibody specific for a polypeptide product of a gene identified by the method of claim 1, the method comprising:
cloning a polynucleotide fragment comprising a sequence of the cloned gene in an expression vector;
isolating a polypeptide expressed by the vector, wherein the polypeptide comprises an amino acid sequence corresponding to the cloned polynucleotide;
immunizing an animal with the isolated polypeptide; and
isolating anti-peptide antibodies specific for the isolated polypeptide from the immunized animal.

33. An isolated novel polynucleotide comprising a gene whose expression level is associated with a disease state, the polynucleotide comprising a nucleic acid sequence encoding a protein comprising a physical characteristic, wherein the polynucleotide or a fragment thereof is differentially expressed in a diseased tissue sample as compared to a normal tissue sample.

34. The isolated polynucleotide of claim 33, comprising a nucleic acid sequence which encodes a protein comprising at least one of the characteristics of: (a) seven transmembrane (7-TM) domains, (b) an amino acid sequence comprising an Asp-Arg-Tyr (DRY) motif and (c) a signal peptide.

35. The isolated polynucleotide of claim 33, wherein the polynucleotide comprises a detectable label.

36. The isolated polynucleotide of claim 33, wherein the polynucleotide, or fragment thereof, is attached to a solid support.

37. The isolated polynucleotide of claim 33, wherein the polynucleotide is single stranded.

38. The isolated polynucleotide of claim 33, wherein the polynucleotide is double stranded.

39. A host cell, comprising the isolated polynucleotide of claim 33.

40. An array comprising at least two polynucleotides according to claim 33.

41. A composition, comprising a test cell sample and an isolated polynucleotide according to claim 33.

42. An electronic library comprising at least one isolated polynucleotide according to claim 33.

43. An isolated polynucleotide comprising:
(a) a polynucleotide having sequences shown in FIGS. 1B, 2B, 3B, 4B and 5B (SEQ ID NOS 1-5), or its complement;
(b) a fragment of the polynucleotide having the sequence shown in FIGS. 1B, 2B, 3B, 4B and 5B (SEQ ID NOS 1-5), or its complement, wherein the fragment is at least 10 nucleotides in length; or
(c) a polynucleotide that selectively hybridizes to the sequences shown in FIGS. 1B, 2B, 3B, 4B and 5B (SEQ ID NOS 1-5) or the fragment in (b), wherein expression of the isolated polynucleotide correlates to a state of disease.

44. The isolated polynucleotide of claim 43, comprising a segment of up to at least 20, 30, 40, 50, 60, 70, 80, 90, 100, 200, 300, 400, 500, 1000 or 1500 nucleotides in length which corresponds identically to a portion of the sequences shown in FIGS. 1B, 2B, 3B, 4B and 5B (SEQ ID NOS 1-5).

45. The isolated polynucleotide of claim 43, wherein the polynucleotide comprises a detectable label.

46. The isolated polynucleotide of claim 43, wherein the polynucleotide is attached to a solid support.

47. The isolated polynucleotide of claim 43, wherein the polynucleotide is single stranded.

48. The isolated polynucleotide of claim 43, wherein the polynucleotide is double stranded.

49. The isolated polynucleotide of claim 43, wherein expression levels of the polypeptide correlate to a state of Parkinson's disease, Alzheimer's disease, or leukemia.

50. A host cell, comprising the isolated polynucleotide of claim 43.

51. An array comprising at least two polynucleotides according to claim 43.

52. A composition, comprising a test cell sample and an isolated polynucleotide according to claim 43.

53. A kit for diagnosing a disease in a test sample, the kit comprising at least one isolated polynucleotide according to claim 43.

54. An electronic library comprising at least one isolated polynucleotide according to claim 43.

55. An isolated polypeptide, or a fragment thereof, whose expression levels in a tissue correlates to a disease state of the tissue, wherein the polypeptide comprises the amino acid sequence encoded by the polypeptides shown in FIGS. 1C, 2C, 3C, 4C and SC (SEQ ID NOS 6-10), or a fragment thereof.

56. The isolated polypeptide of claim 55, wherein the disease state is selected from the group consisting of central nervous system (CNS) disease, Parkinson's disease, Alzheimer's disease and leukemia.

57. The isolated polypeptide of claim 55, wherein expression levels of the polypeptide correlate to a state of CNS disease, Parkinson's disease, Alzheimer's disease, or leukemia.

58. The isolated polypeptide of claim 55, wherein the polypeptide comprises a fragment that includes an antigenic epitope comprising the amino acid sequence shown in FIGS. 1C, 2C, 3C, 4C and 5C (SEQ ID NOS 6-10).

59. The isolated polypeptide of claim 55, wherein the polypeptide or fragment thereof, is attached to a solid support.

60. An isolated antibody, or antigen binding fragments thereof, that bind to the polypeptide according to any one of claims 55.

61. The isolated antibody of claim 60, wherein the antibody is a polyclonal antibody.

62. The isolated antibody of claim 60, wherein the antibody is a monoclonal antibody.

63. The isolated antibody of claim 60, wherein the antibody is attached to a solid surface.

64. The isolated antibody of claim 60, wherein the antibody comprises a detectable label.
